(19) United States
(12) Patent Application Publication
(10) Pub. No.: US 2012/0245229 A1
(43) Pub. Date: Sep. 27, 2012

(54) METHOD FOR TREATING NEUROPATHIC PAIN

(76) Inventors: Ru-Rong Ji, Needham, MA (US); Charles N. Serhan, Needham, MA (US)

(21) Appl. No.: 13/395,758

(22) PCT Filed: Sep. 15, 2010
(86) PCT No.: PCT/US2010/048879

§ 371 Date: May 29, 2012

Related U.S. Application Data
(60) Provisional application No. 61/276,656, filed on Sep. 15, 2009.
(60) Provisional application No. 61/318,981, filed on Mar. 30, 2010.

(51) Int. Cl. A61K 31/202; A61P 25/00; A61P 29/00

(52) U.S. Cl. 514/560

(57) ABSTRACT

The invention describes a method to treat neuropathic pain and/or inflammatory pain, comprising the step of administering to a subject in need thereof an effective amount of a resolvin, such that neuropathic pain and or inflammatory pain is treated.

### CROSS REFERENCE TO RELATED APPLICATIONS

This application claims benefit under 35 U.S.C. 119(e) to U.S. Ser. Nos. 61/276,656, filed Sep. 15, 2009, entitled “Method for Treating Neuropathic Pain” and 61/318,981, filed Mar. 30, 2010, entitled “Resolvins RvE1 and RvD1 Attenuate Inflammatory Pain via Central and Peripheral Actions”, the contents of both are incorporated herein by reference in their entirety.

### STATEMENT REGARDING FEDERALLY SPONSORED RESEARCH

The work leading to this invention was supported in part by NIH R01-DE17794, R01-N554362, R37 GM38765, R01-DE019938, and R01-DK074448. The U.S. Government therefore may have certain rights in the invention.

### FIELD OF THE INVENTION

The invention relates generally to the use of resolvins, resolvin analogs, protectins, protectin analogs, and analogues of lipid mediators derived from omega-3 polyunsaturated fatty acids (PUFAs) to treat neuropathic pain and/or pain associated with inflammation.

### BACKGROUND OF THE INVENTION

More than 30 million Americans suffer from unrelieved chronic pain, such as nerve injury-induced neuropathic pain, which is regarded as a disease with its own pathology. Although a considerable amount is known about how chronic pain is induced, little is known about how acute pain naturally resolves. Current management of chronic pain mainly focuses on two types of drugs, ones that treat pain symptoms by blocking neurotransmission and those that modify disease progression by suppressing neuroinflammation.

It is widely believed that chronic pain results from neural plasticity that occurs both in the peripheral nervous system [PNS, e.g., dorsal root ganglion (DRG)] and central nervous system (CNS, e.g., spinal cord). Therefore, drug development for pain therapy has been largely focused on neuronal targets to block neural transmission or sensitization with opioids and ion channel blockers. However, these drugs only produce transient pain relief during medication and often produce significant side effects. Recent progress indicates that immune and glial regulation plays a powerful role in chronic pain development and maintenance. After peripheral nerve injury, immune cells such as macrophages invade the damaged nerve and DRG and remain there for months and even years. Nerve injury-induced activation of glial cells (e.g., microglia and astrocytes) in the spinal cord can also persist for a very long period. Immune and glial cells are involved in neuroinflammation in the PNS and CNS, producing various inflammatory mediators such as prostaglandins, leukotrienes, cytokines, chemokines, and matrix metalloproteases. Inhibiting the action of these pro-inflammatory mediators reduces pain symptoms and attenuates neuroinflammation. However, some anti-inflammatory drugs such as cyclooxygenase (COX) and lipoxygenase (LOX) inhibitors delay the resolution of inflammation and tissue's return to homeostasis, because these enzymes are also required for the production of pro-resolving mediators. Ion channel blocker such as lidocaine also inhibits the resolution program.

It is often ignored that tissue injury not only produces proinflammatory mediators but also generates anti-inflammatory and pro-resolving mediators, resulting in resolution of acute inflammation and acute pain. Resolution of acute inflammation, once thought to be a passive process, is now shown to involve active biochemical programs that enable inflamed tissues to return to homeostasis. Accumulating evidence indicates that anti-inflammation and pro-resolution are distinct mechanisms in the control of inflammation. The actions of pro-resolution mediators are in sharp contrast to those of currently used anti-inflammatory therapeutics (e.g., inhibitors of COX and LOX), which are inhibitors of resolution. Disruption of acute resolving processing will lead to uncontrolled inflammation that is implicated in the pathogenesis of many chronic diseases.

Therefore, a need exists for compositions and methods that overcome one or more of the current disadvantages noted above.

### BRIEF SUMMARY OF THE INVENTION

The present invention surprisingly provides compositions and methods that reduce, eliminate or prevent pain associated with inflammation and/or neuropathic pain. The compositions include resolvin and protectin compounds that occur from physiological release, resolvin and protectin analogs that have been derivativized by inclusion of additional functionality not generally associated with a naturally occurring resolvin (such as an ethyl or methyl ester thereof), and analogues of lipid mediators derived from omega-3 polyunsaturated fatty acids (PUFAs), herein collectively referred to as “resolvins”. The present invention provides that disruption of local active pro-resolving processing will result in chronic pain and that the resolvins described herein can reduce, eliminate or prevent such pain.

The resolvins described herein are effective, in nanogram dose range, in producing anti-hyperalgesic effects of inflammatory pain and neuropathic pain.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1. Preemptive spinal (intrathecal) administration of RvE1, at very low doses, reduces formalin-induced inflammatory pain in the 2^{nd} phase. (a, b) Reduction of formalin-induced spontaneous pain in the 2^{nd} phase by RvE1, morphine, and the COX-2 inhibitor NS-398. a, time course. *P<0.05 (vehicle vs RvE1). b, 1^{st}- and 2^{nd}-phase. *P<0.05, vs vehicle, n=5-8. (c) Dose response curve of percentage inhibition (vs vehicle control) of RvE1, morphine, and NS-398 on formalin-induced pain in the 2^{nd} phase. (d) Requirement of Gai but not opioid receptor for RvE1-mediated inhibition of 2″ phase pain. PTX, pertussis toxin; N.S., no significance. *P<0.05, n=5-7. (e) Dose-dependent reduction of 2^{nd} phase pain by the ChemR23 agonist chemerin. *P<0.05, vs vehicle, n=6. (f) Expression of ChemR23 mRNA in the DRG and spinal cord dorsal horn, as revealed by in situ hybridization. Scales, 50 μm. (g) Co-localization of ChemR23 with TRPV1 in a cultured DRG neuron (upper) and with NeuN in the superficial dorsal horn (lower), as demonstrated by double immunostaining Scales, 25 μm.

FIG. 2. Central and peripheral actions of resolvins on persistent inflammatory pain and inflammation. (a-d) Attenuation of complete Freund's adjuvant (CFA)-evoked heat hyperalgesia by intrathecal administration of resolvins given on post-CFA day 3. a. Development of heat hyperalgesia 3 days after CFA injection. b. Acute actions (15-45 min) of RvE1, RvD1, DHA, and EPA. c. Persistent actions (1-6 h) of RvE1, NS-398, and 19-pf-RvE1, a modified form of RvE1. d. Lack of effects of RvE1 on basal pain thresholds in naïve mice. PWL, paw withdrawal latency; M.P.E, maximum possible effect of anti-hyperalgesia. *P<0.05, vs baseline (BL, a) or vehicle (b-d); ^{#}P<0.05, n=4-7. (e-h) Reduction of carrageenan (CRG)-elicited heat hyperalgesia (e), paw edema (f), neutrophil infiltration (g), and expression of proinflammatory cytokines and chemokines (h) in the inflamed paw, following intraplantar pretreatment of resolvins. Edema, neutrophil infiltration, and cytokine expression at protein levels were examined by paw volume (f), myeloperoxidase (MPO) activity (g), and cytokine array (h), respectively, at 4 or 2 h (h) after CRG injection. *P<0.05, vs vehicle (e, f) or naïve (g, h), ^{#}P<0.05, vs CRG (h), n=3-6.

FIG. 3. Spinal administration of RvE1 reduces heat hyperalgesia and spontaneous pain by blocking TRPV1 and TNF-α signaling in DRG neurons and spinal presynaptic terminals. (a) Reduction of CFA-induced heat hyperalgesia and loss of formalin-induced 2^{nd} phase pain in Tnfr^{-/-} mice (double knockout mice of both Tnfr1 and Tnfr2). (b) Loss of intrathecal TNF-α-induced heat hyperalgesia but not formalin-induced 2^{nd} phase pain in Trpv1^{-/-} mice. (c) Prevention of intrathecal TNF-α-induced heat hyperalgesia by RvE1. * P<0.05, vs wild-type control (a, b) or vehicle (c), n=4-6. (d) Inhibition of TNF-α-evoked sEPSC frequency increase by perfusion of RvE1 and capsazepine (CZP, 10 μM) in spinal cord lamina II neurons. Low panel, quantification of sEPSC frequency and amplitude. (e) Inhibition of capsaicin-evoked sEPSC frequency increase by RvE1, in a pertussis toxin (PTX, 0.5 μg/ml)-sensitive manner, and by MEK inhibitor PD98059 and U0126 (1 μM) in lamina II neurons. Low panels (d, e), quantification of sEPSC frequency and amplitude. *P<0.05, vs baseline; ^{#}P<0.05, vs TNF-α (d) or capsaicin (e); ^{$}P<0.05; N.S., not significant, n=5-10 neurons. (f) Prevention of intrathecal capsaicin-evoked acute spontaneous pain by RvE1. *P<0.05, vs RvE1, n=6. (g) Inhibition of TNF-α and capsaicin-evoked ERK phosphorylation (pERK) in cultured DRG neurons by RvE1, in a PTX-sensitive manner. Scale, 100 μm. Low panel, percentage of pERK-positive neurons in DRG cultures. *P<0.05, n=4. (h) Schematic of RvE1-induced inhibition of inflammatory pain (heat hyperalgesia) via presynaptic mechanisms.

FIG. 4. Spinal RvE1 administration attenuates mechanical allodynia and blocks TNF-α signaling in postsynaptic dorsal horn neurons. (a, b) Reduction of intrathecal TNF-α-induced mechanical allodynia by RvE1 pretreatment (a) but not in Trpv1^{-/-} mice (b). *P<0.05, vs vehicle control, n=5. (c) Blockade of TNF-α-evoked increase of NMDA currents by RvE1 in lamina II neurons. *P<0.05, vs baseline, ^{#}P<0.05, n=6. (d) Inhibition of TNF-α-induced ERK phosphorylation (pERK) in superficial dorsal horn neurons by RvE1. White line, border of the dorsal horn gray matter. *P<0.05, n=4. (e) Blockade of TNF-α-induced increase of NMDA current by MEK inhibitor PD98059 (1 μM) and U0126 (1 μM) but not by capsazepine (CZP, 10 μM). *P<0.05, vs corresponding baseline; ^{#}P<0.05, vs TNF-α; n=5-10 neurons. (f) Schematic of RvE1-induced inhibition of inflammatory pain (mechanical allodynia) via postsynaptic mechanisms.

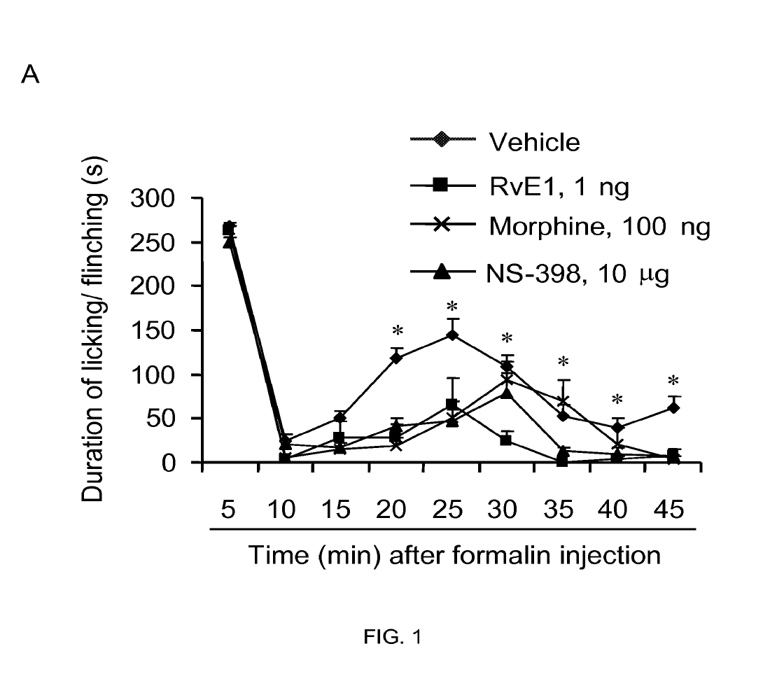

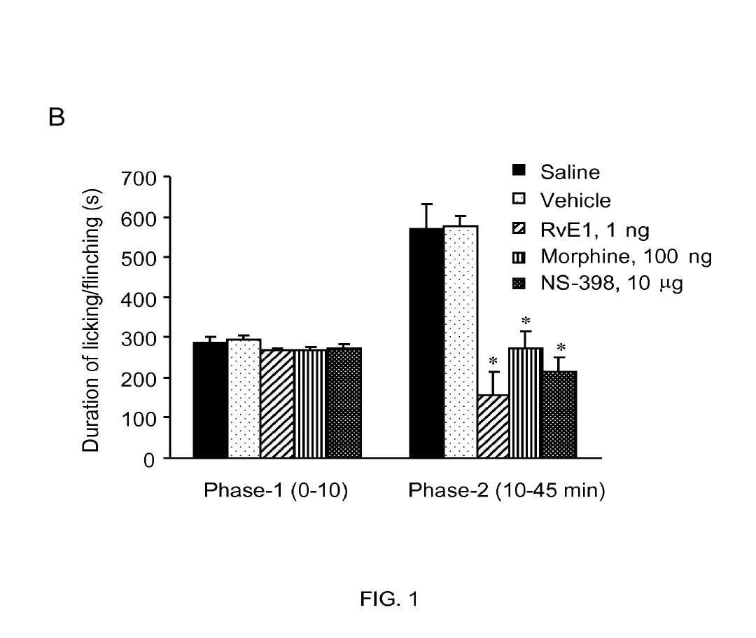

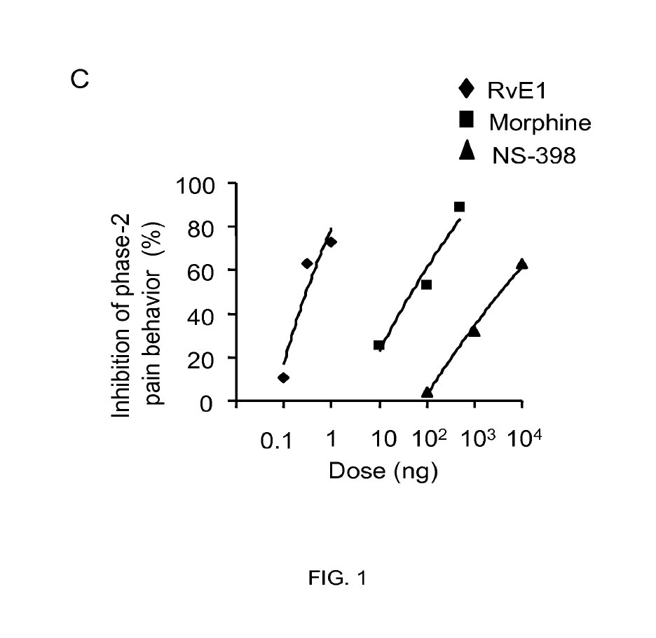

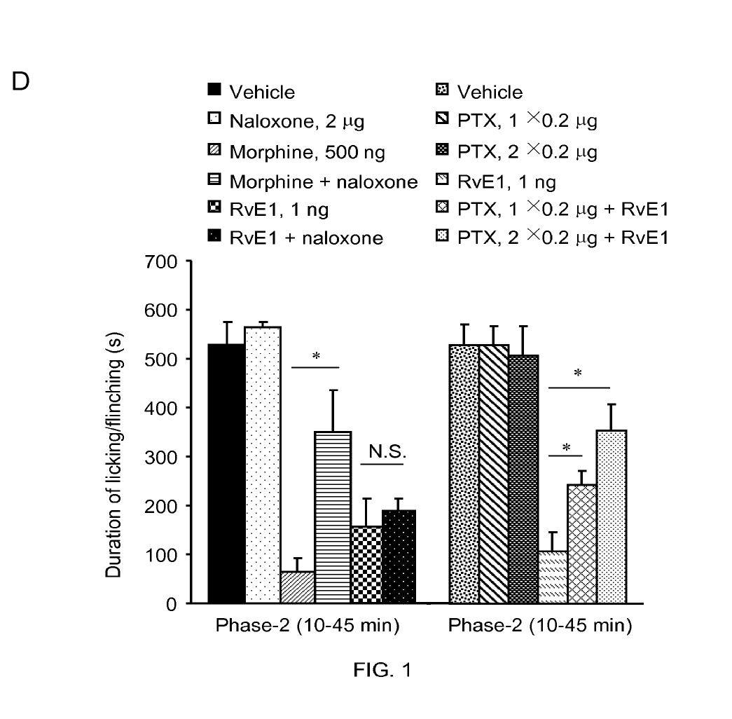

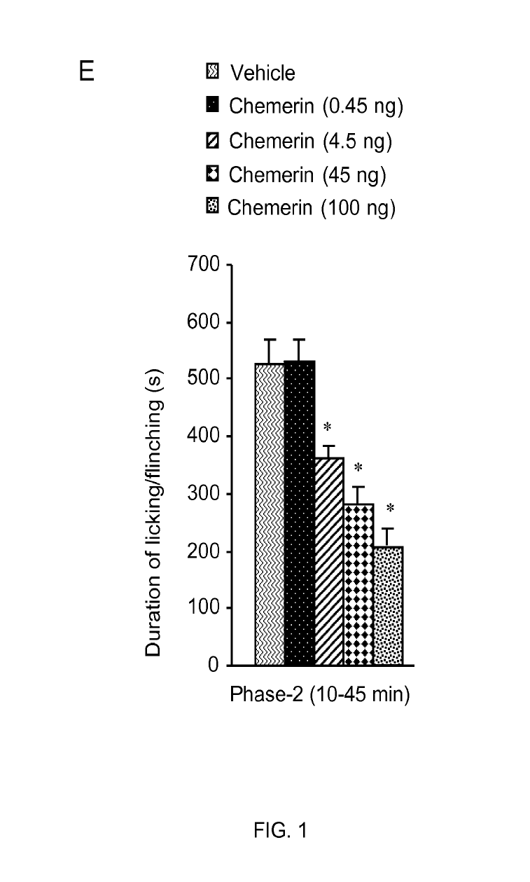

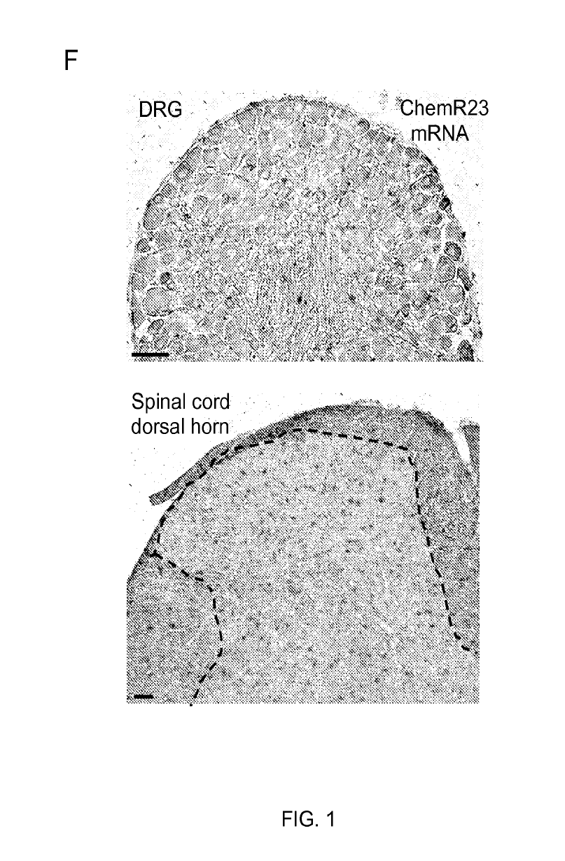

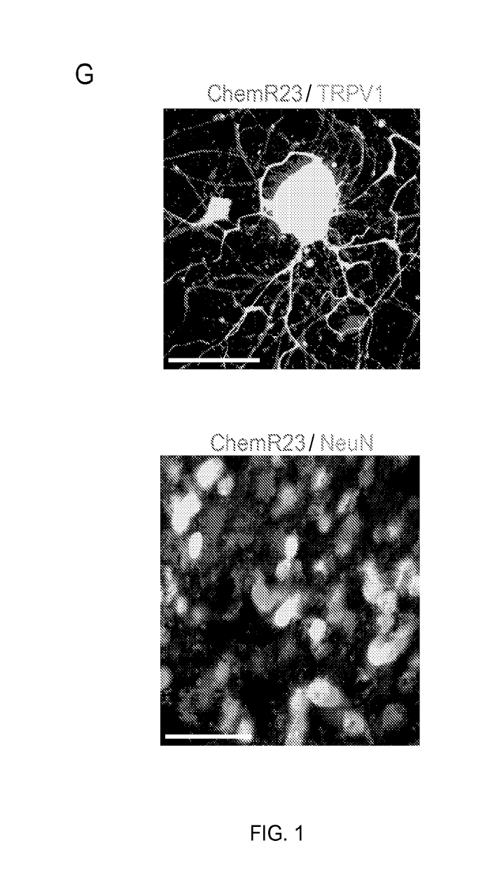

Supplementary FIG. 1. Reversal of RvE1-induced antinociceptive effects by ChemR23 siRNA treatment. (a, b) Intrathecal injections of ChemR23 siRNA (3 μg, daily for 3 days, before formalin injection) reduces ChemR23 expression in the DRG (a) and reverses RvE1-induced antinociceptive effect in the 2^{nd}-phase of formalin test (b). DRG tissues were collected after formalin test and processed for ChemR23 western blotting. Note the 1^{st}-phase pain is not affected. *P<0.05, n=4-7. (c, d) Intraplantar injections of ChemR23 siRNA (4 μg, delivered at 48 h, 24 h, and 3 h before carrageenan injection) reduces ChemR23 expression in the hindpaw skin (c) and reverses RvE1-induced anti-hyperalgesic effect at 4 h after the carrageenan (CRG) inflammation (d). Skin tissues were collected after behavioral testing and processed for ChemR23 western blotting. * P<0.05, n=3-5.

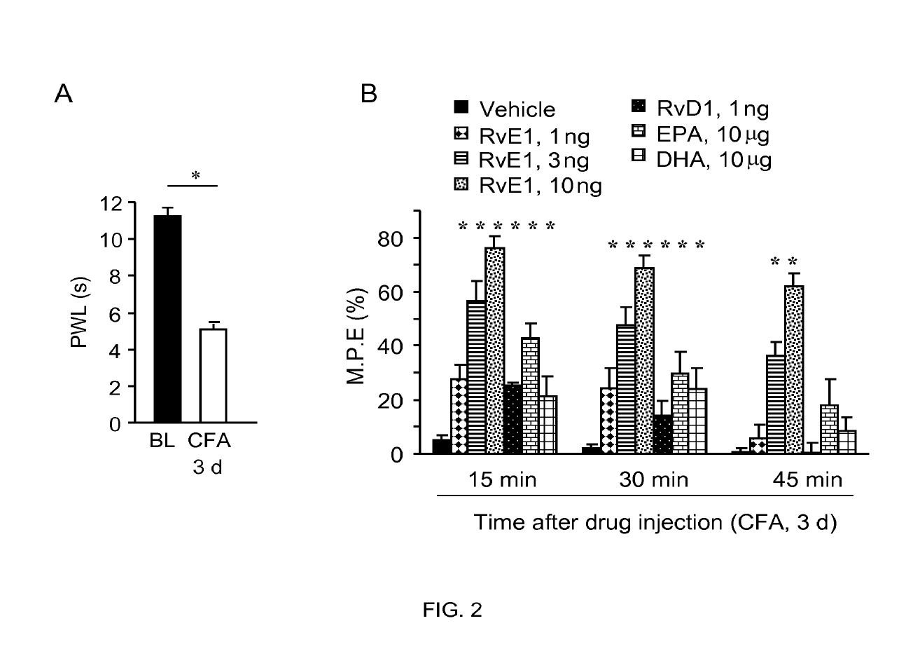

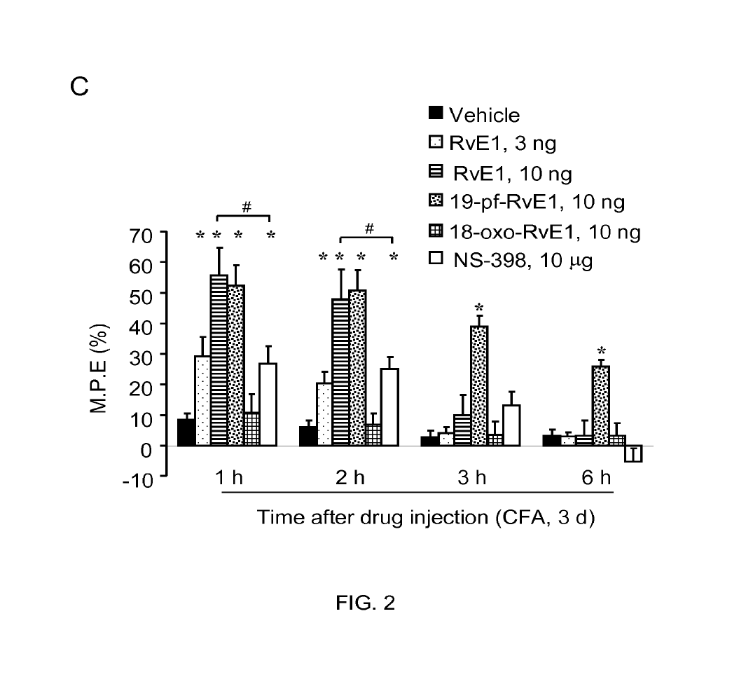

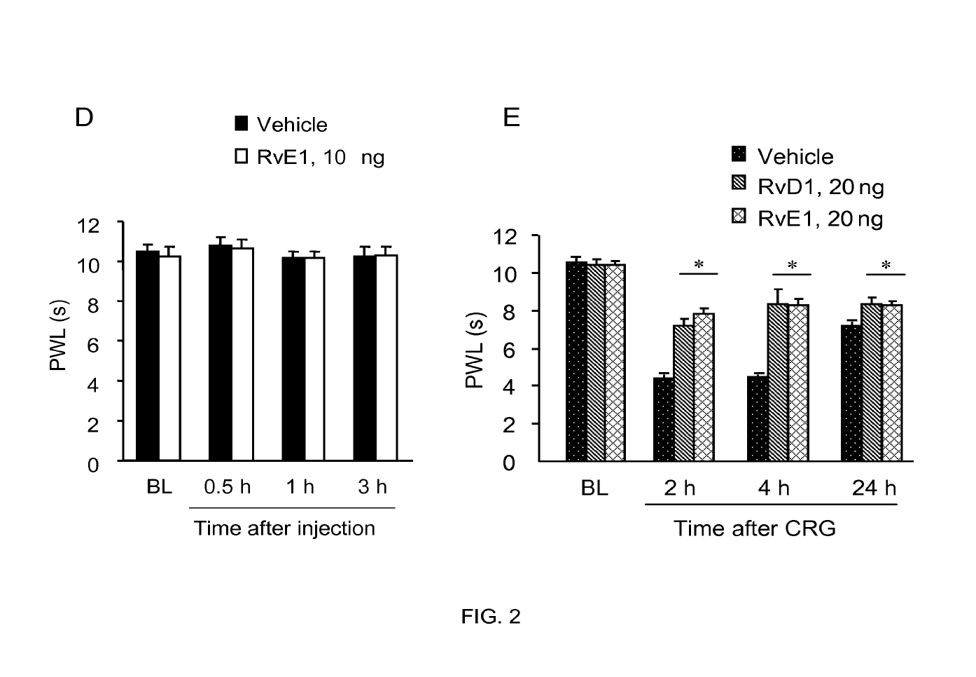

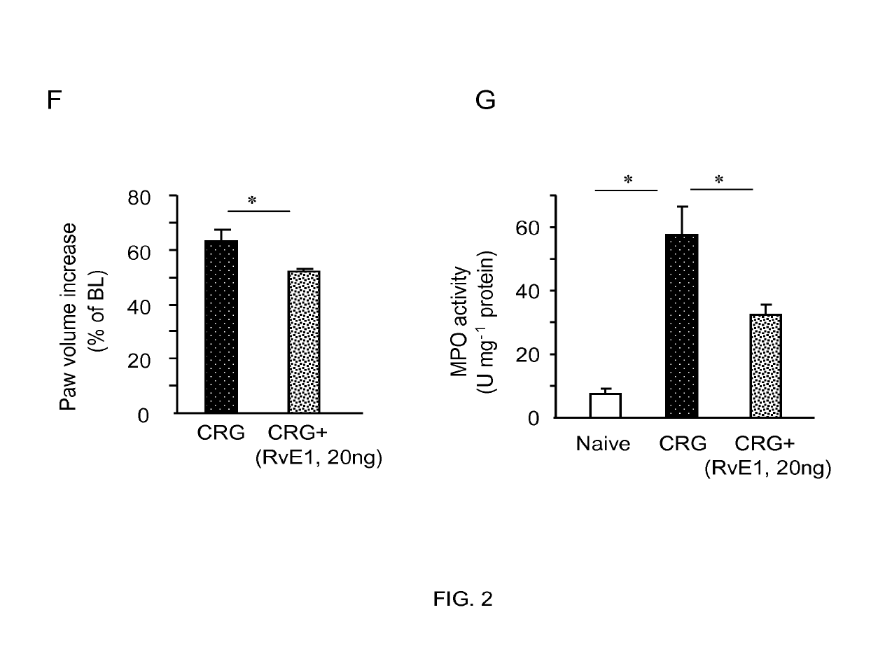

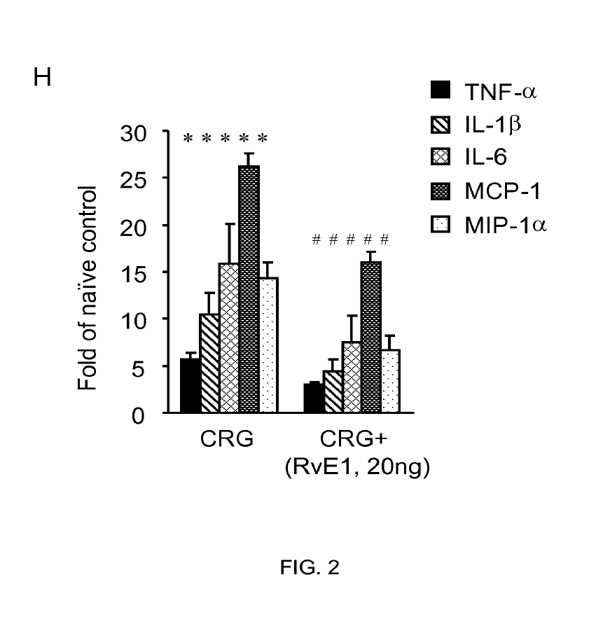

Supplementary FIG. 2. ChemR23 is expressed in DRG neurons that co-express TRPV1. (a-c) Double staining of immunohistochemistry in DRG sections shows co-localization of ChemR23 and TRPV1. b, high magnification of a in the box. c, another example of co-localization using TSA amplification. Red, green and yellow arrows indicate ChemR23-labeled, TRPV1-labeled, and double-labeled neurons, respectively. (d) Quantification of ChemR23-, TRPV1-, and double-labeled neurons in DRG sections. Note that more than 60% TRPV1+ neurons express ChemR23. (e) Double staining of immunocytochemistry in DRG cultures shows co-localization of ChemR23 with TRPV1 in dissociated DRG neurons. Scales, 50 μm.

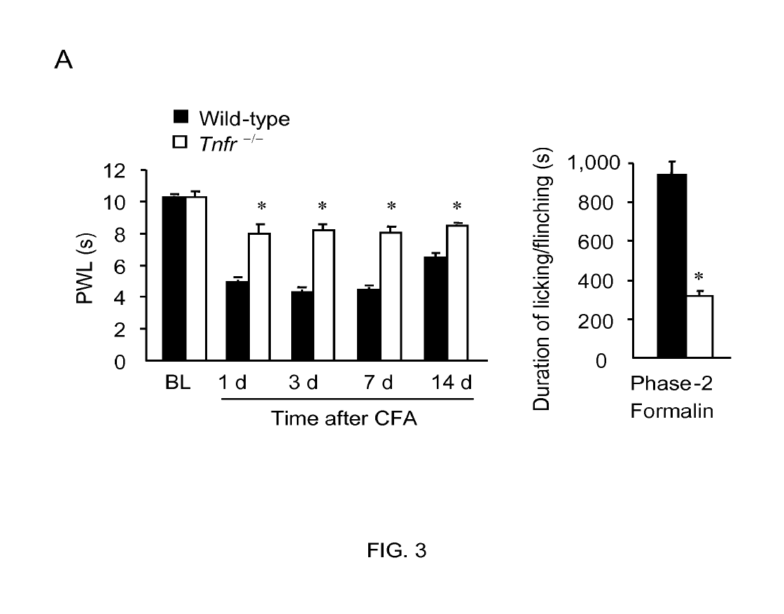

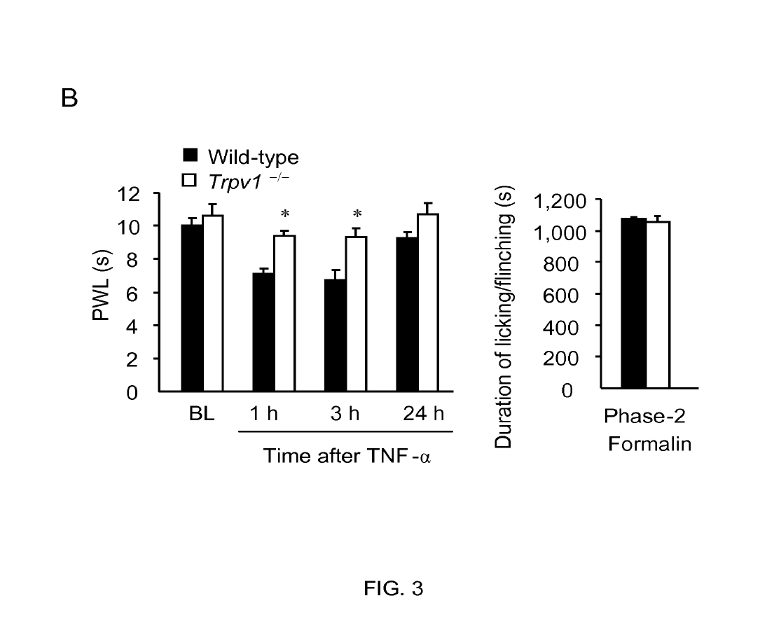

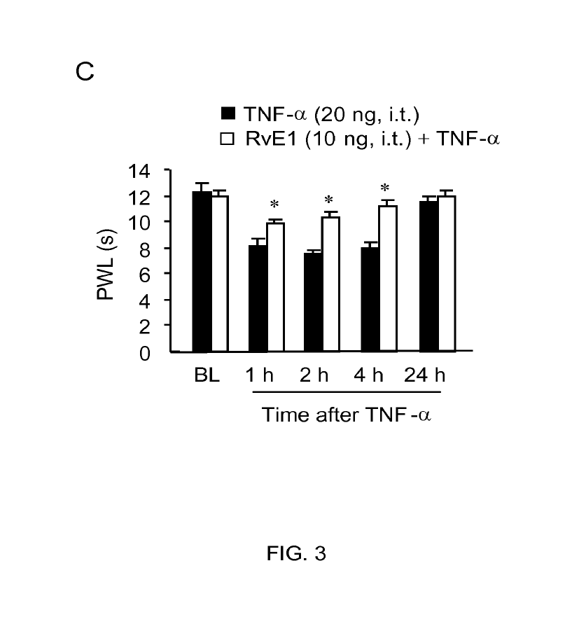

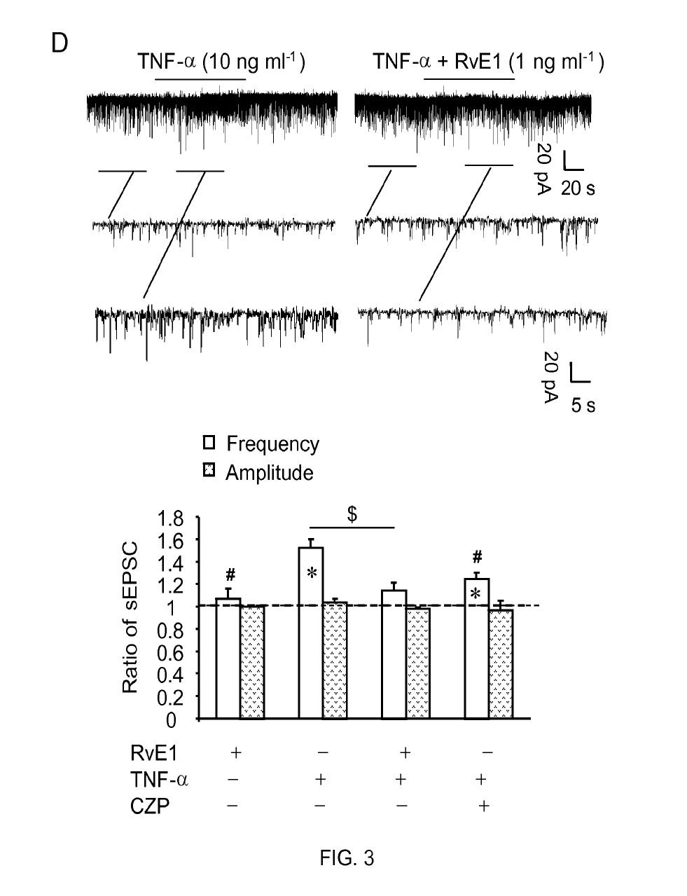

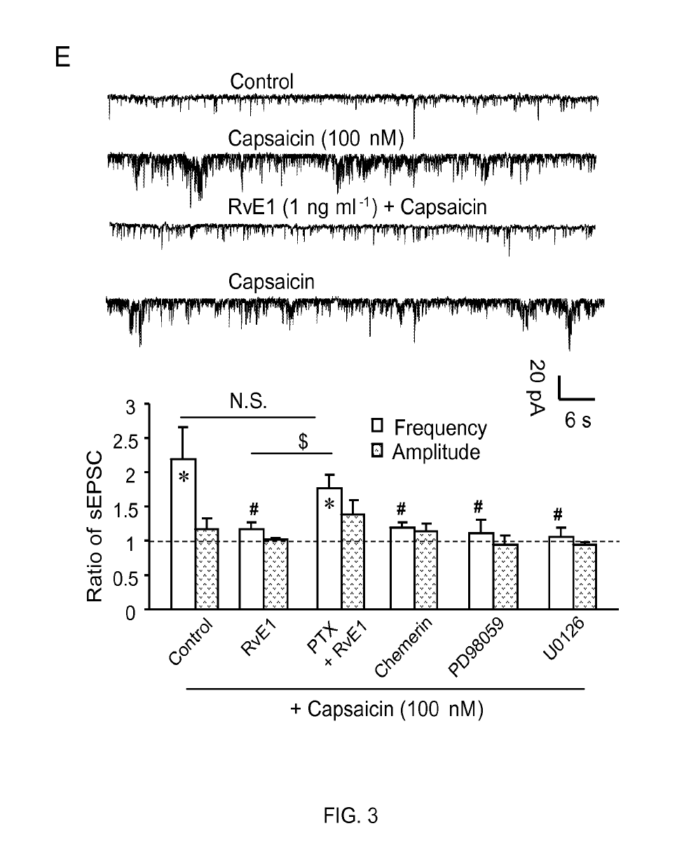

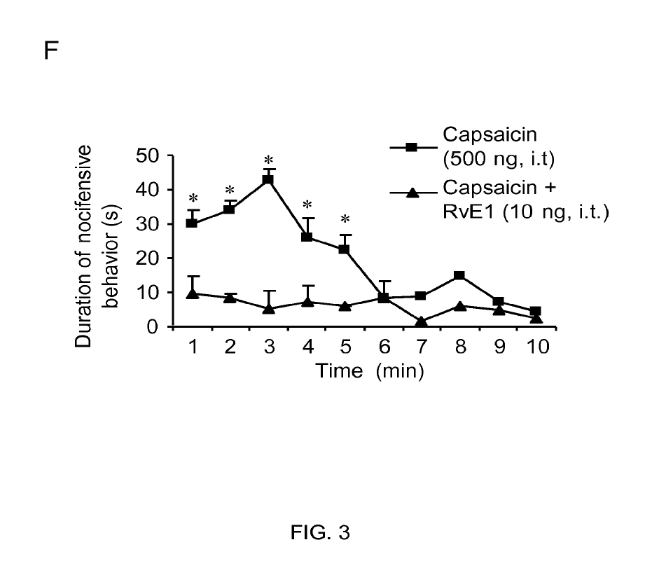

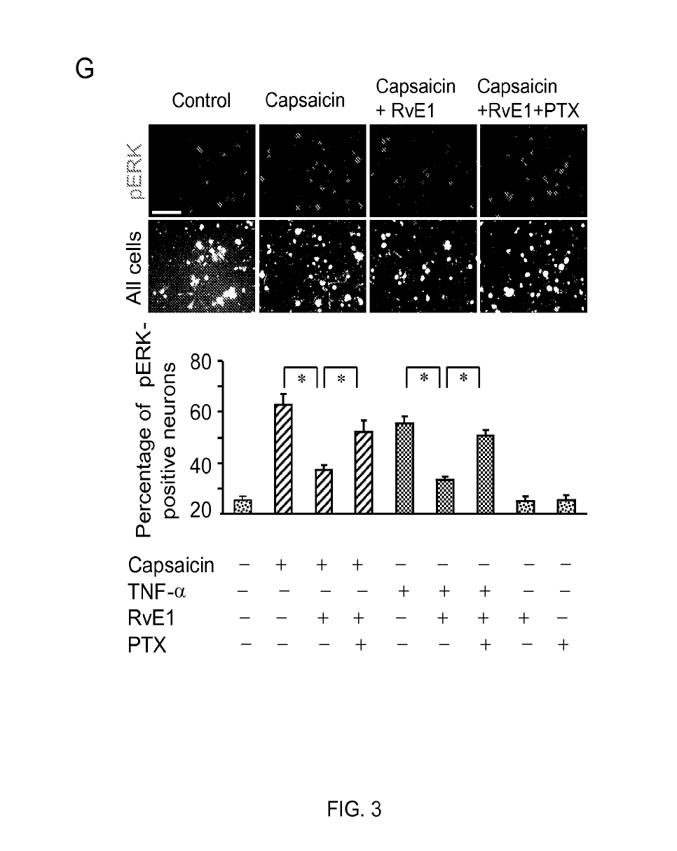

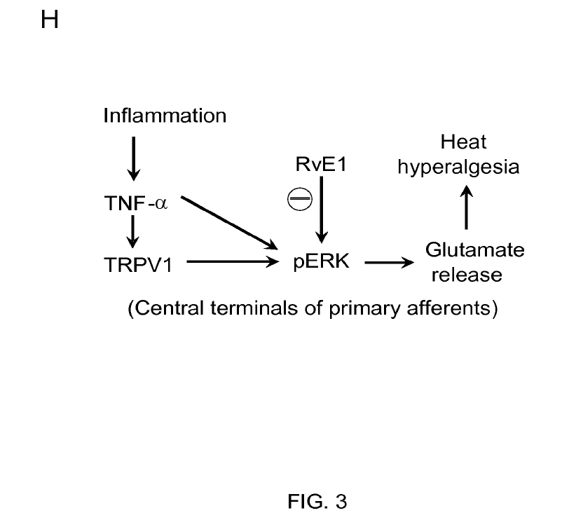

Supplementary FIG. 3. ChemR23 is expressed in dorsal horn neurons and central terminals of primary afferents in the superficial spinal cord. (a, b) Double immunostaining shows co-localization of ChemR23 with NeuN (neuronal marker) in dorsal horn neurons of the spinal cord. b, 3-panel images showing colocalization (box in a). (c) Lack of ChemR23 immunostaining in the dorsal horn after preabsorption of primary antibody with a blocking peptide. (d) Co-localization of ChemR23 and substance P(SP) in central axonal terminals of primary afferents in the superficial dorsal horn. Upper left, low magnification of ChemR23 in the dorsal horn. Lower left, high magnification from the box. Note SP-labeled terminals are enriched in lamina I. Yellow arrows indicate double-labeled terminals. Scales, 50 μm.

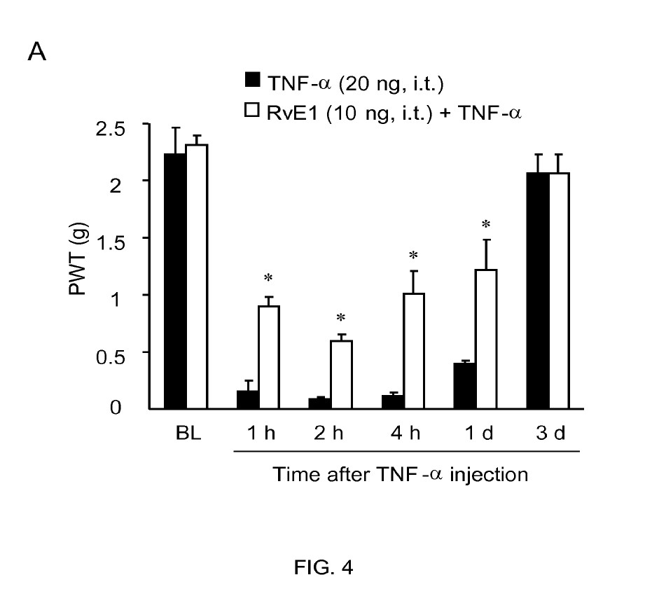

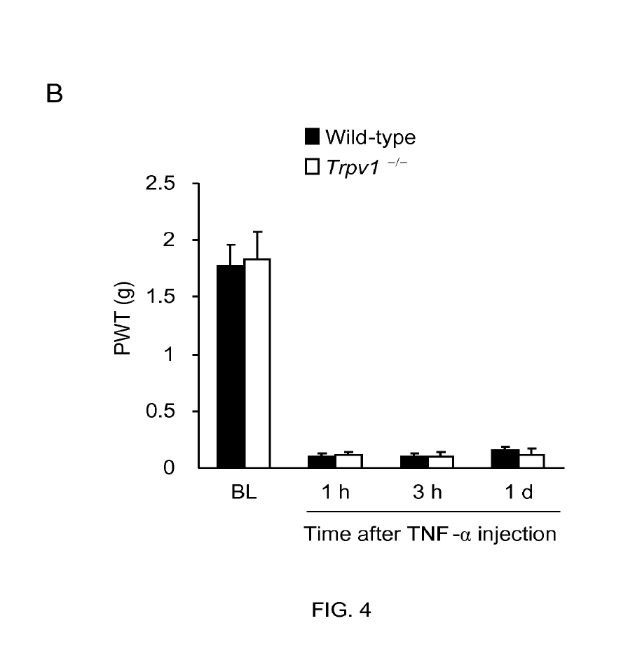

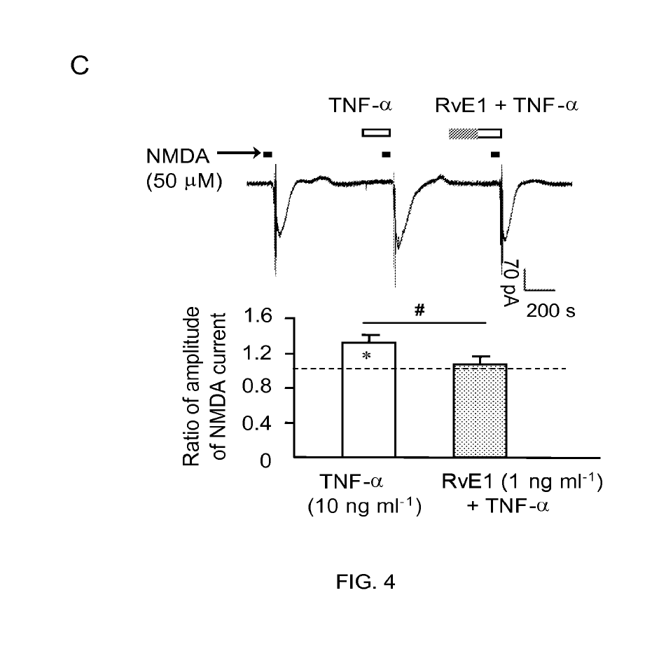

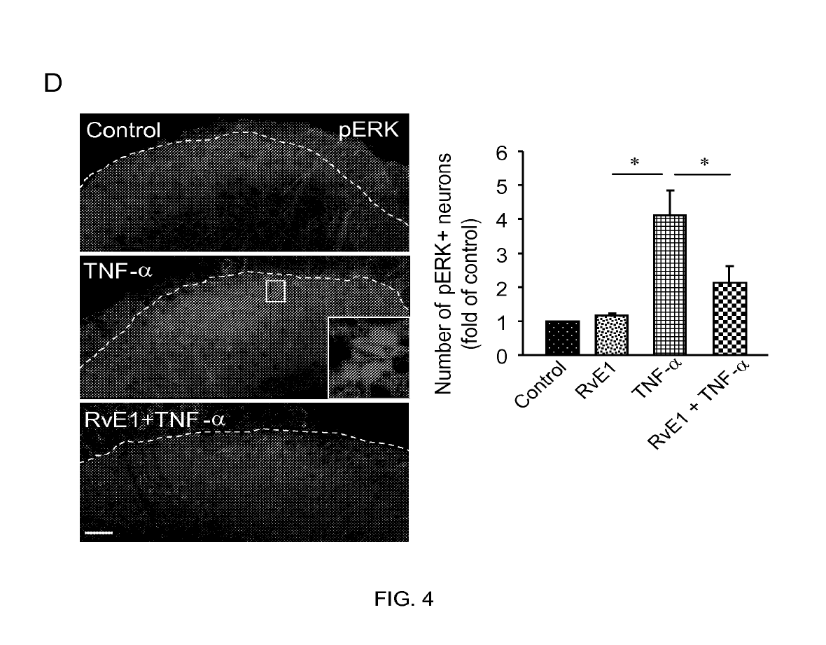

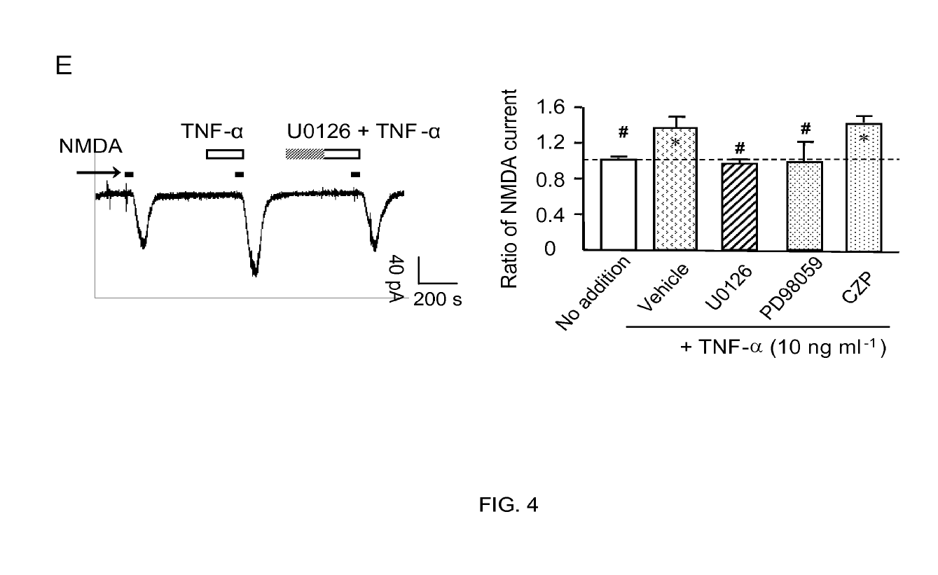

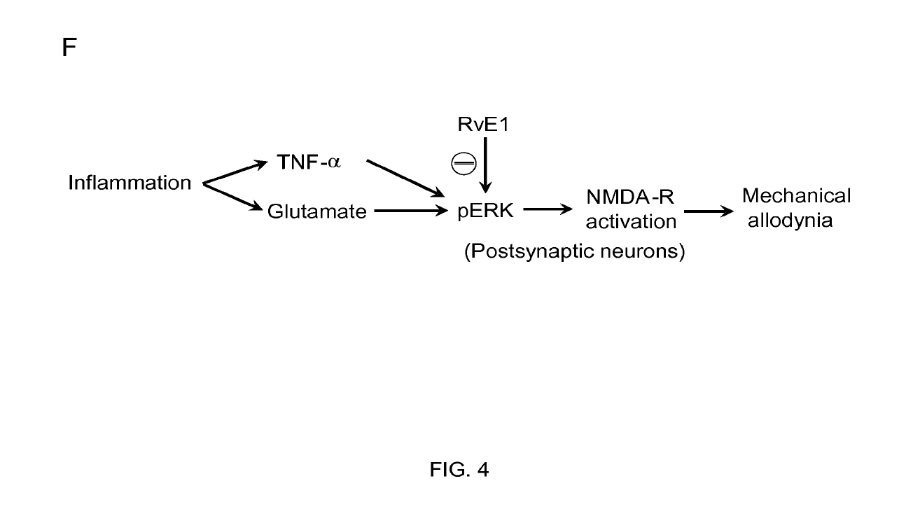

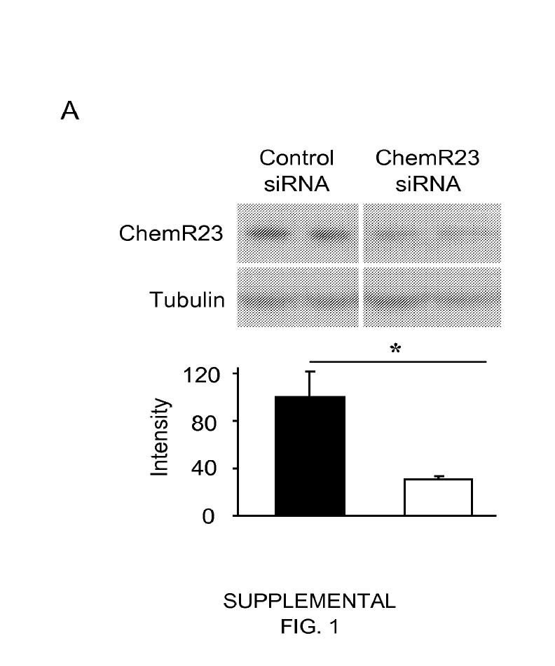

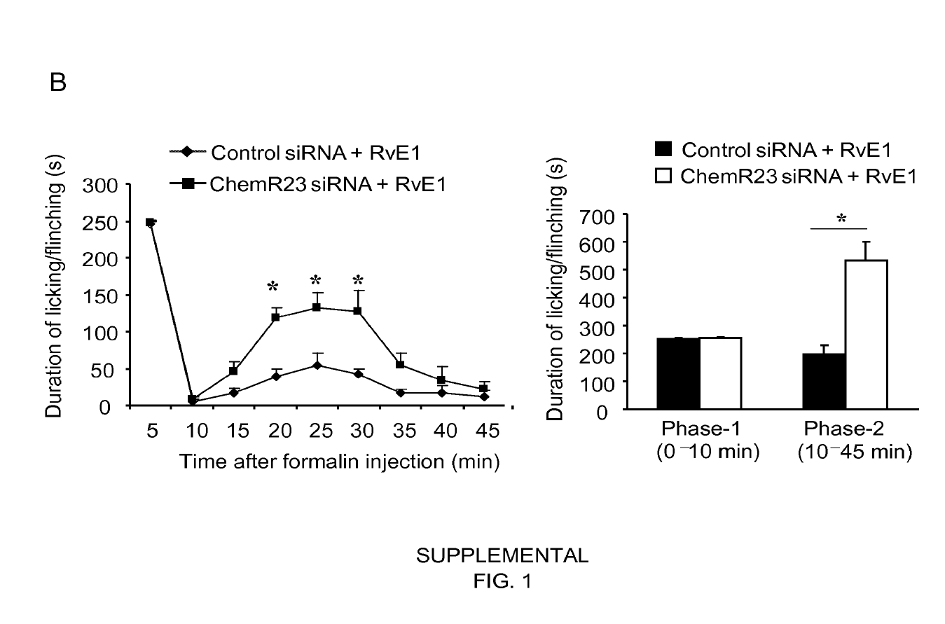

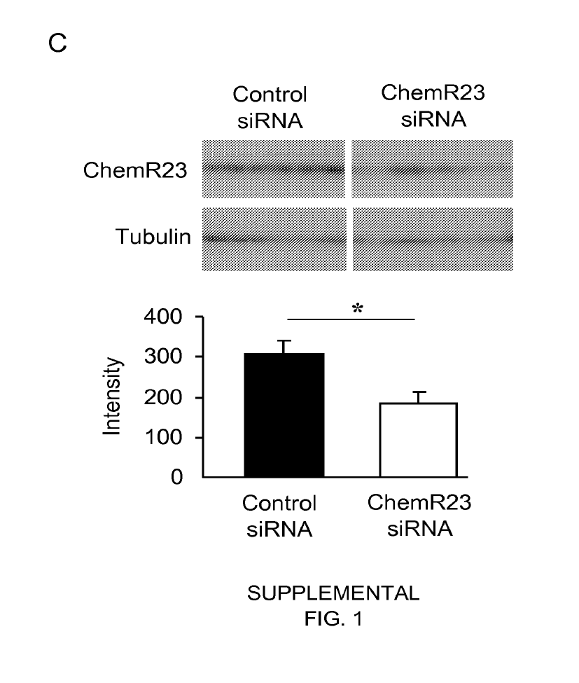

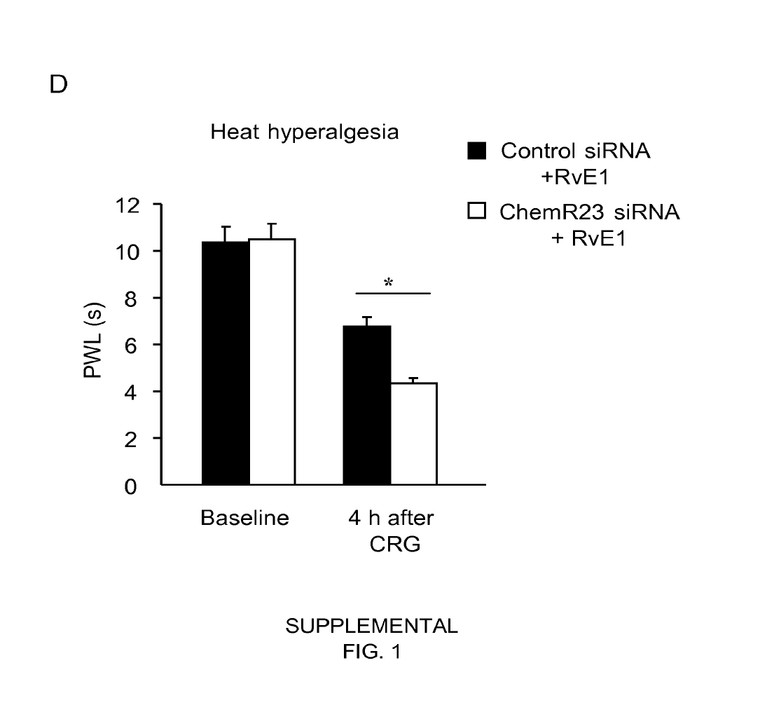

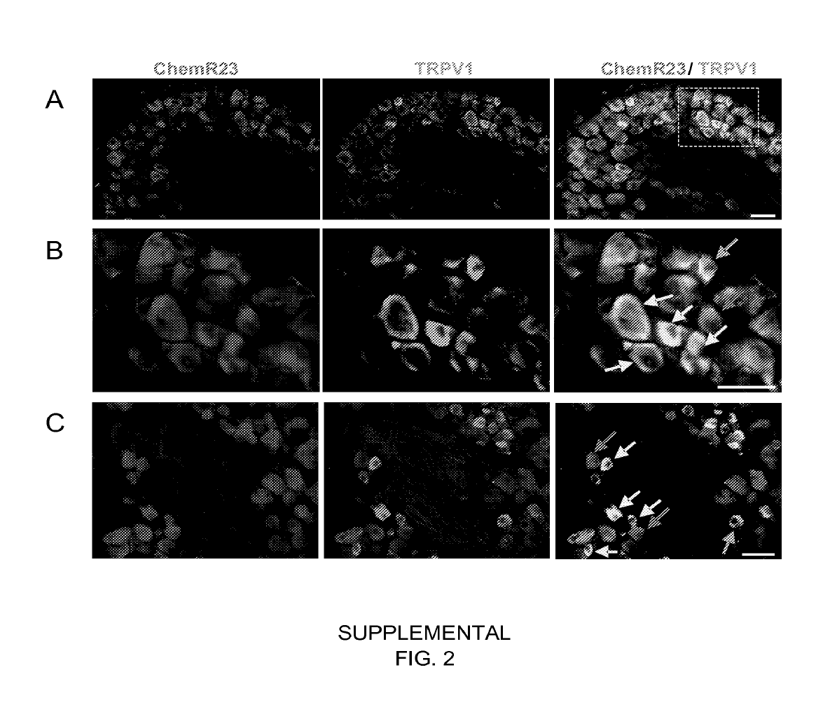

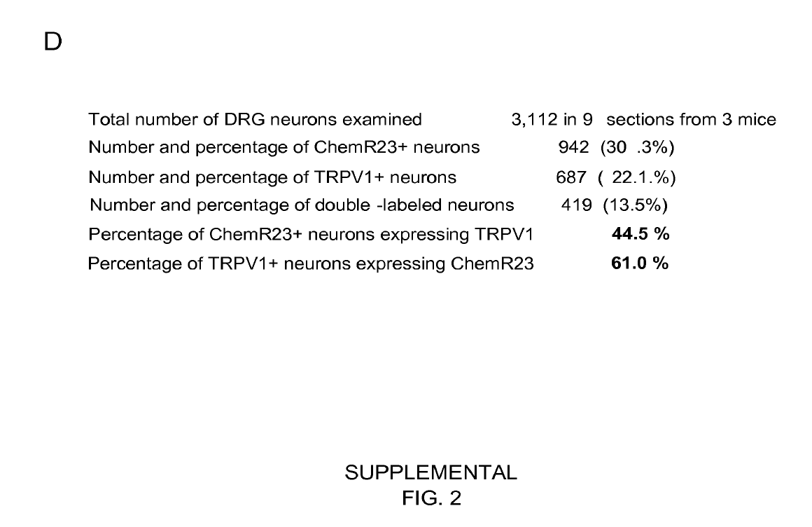

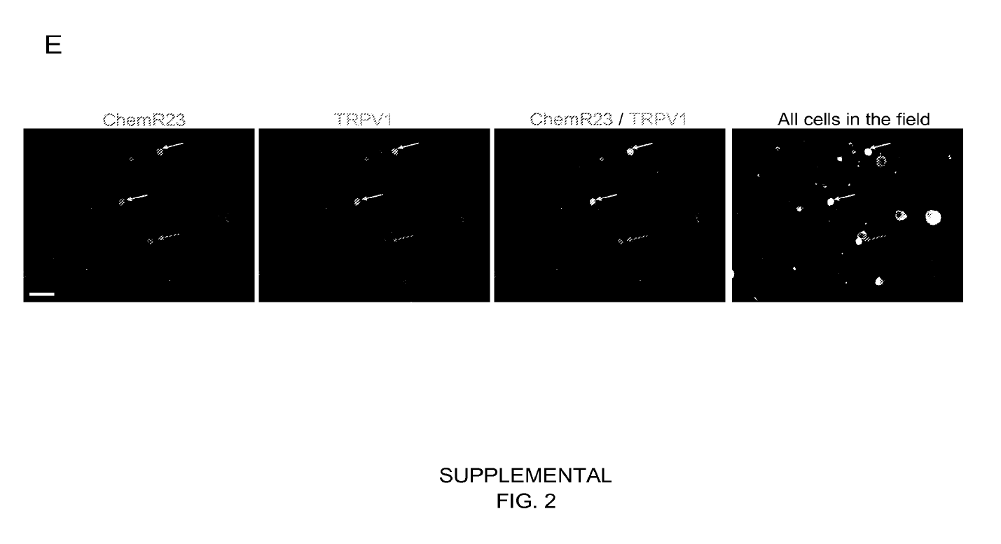

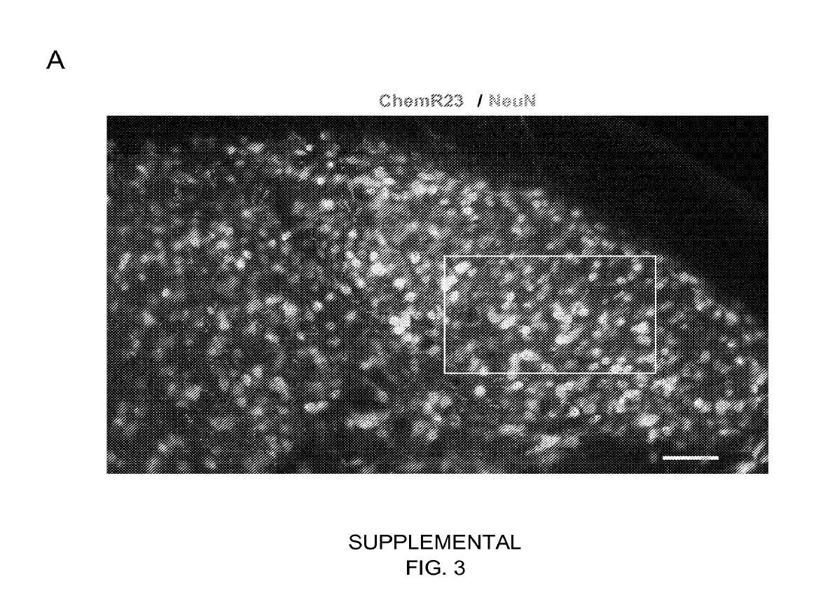

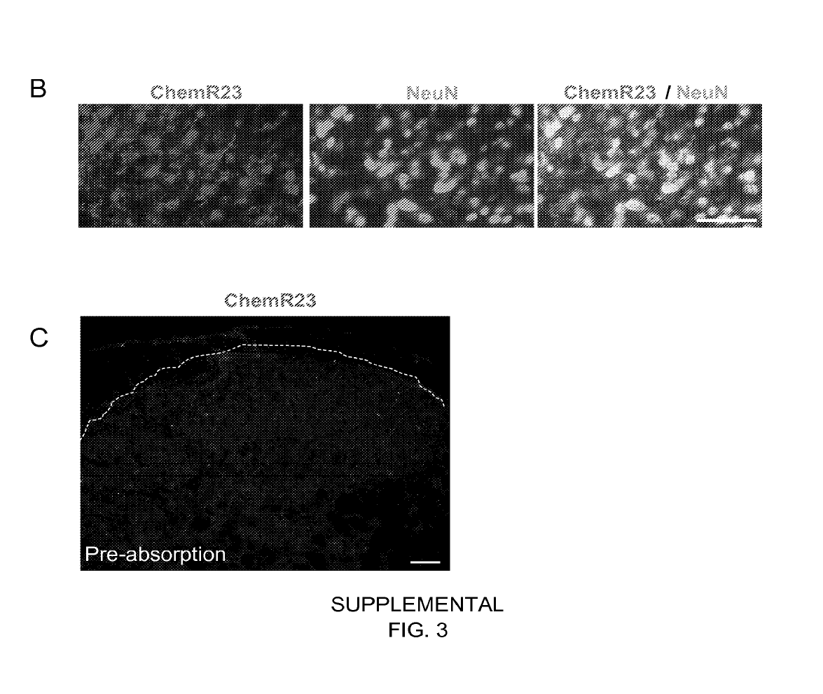

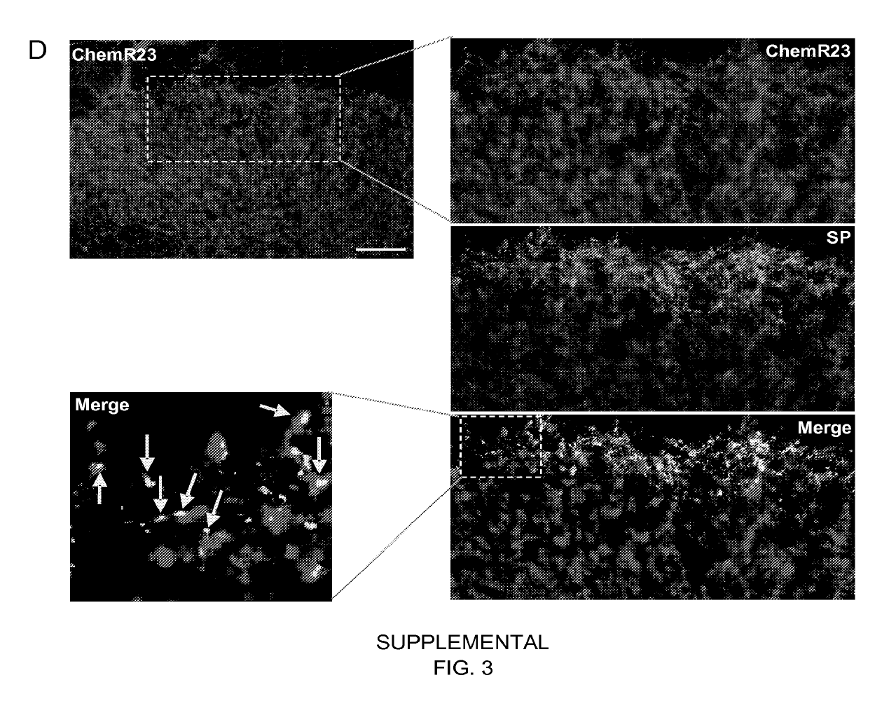

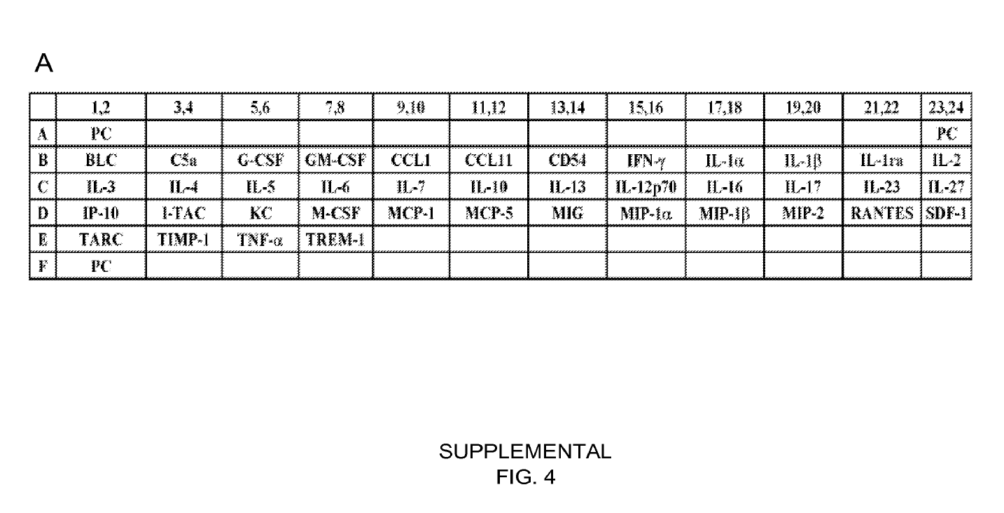

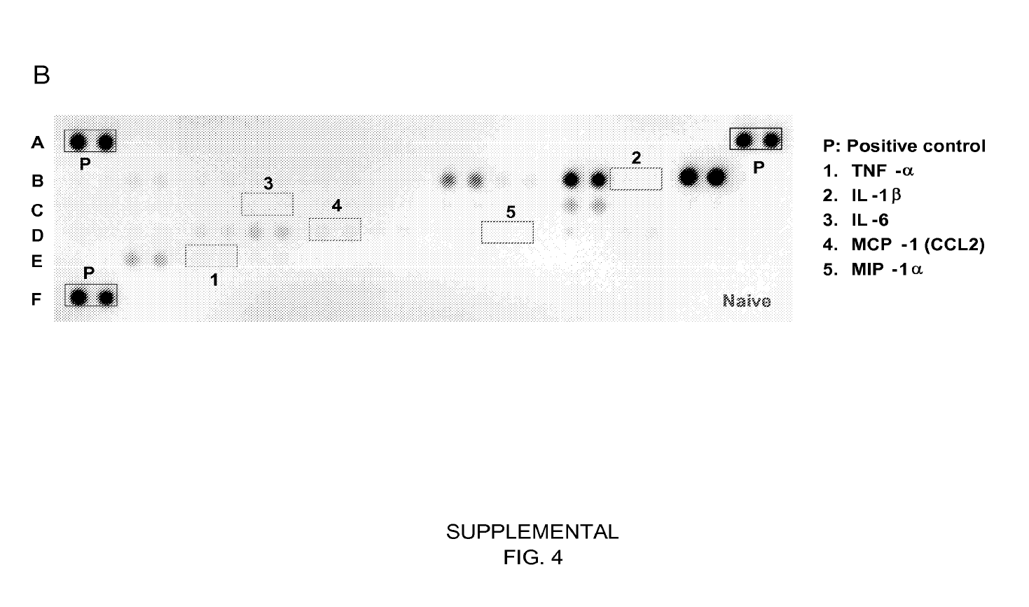

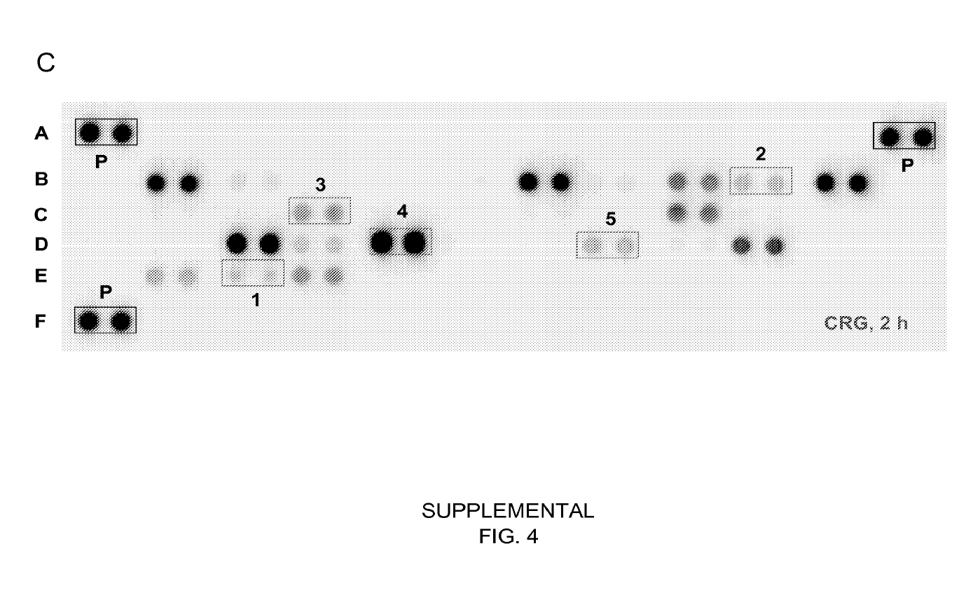

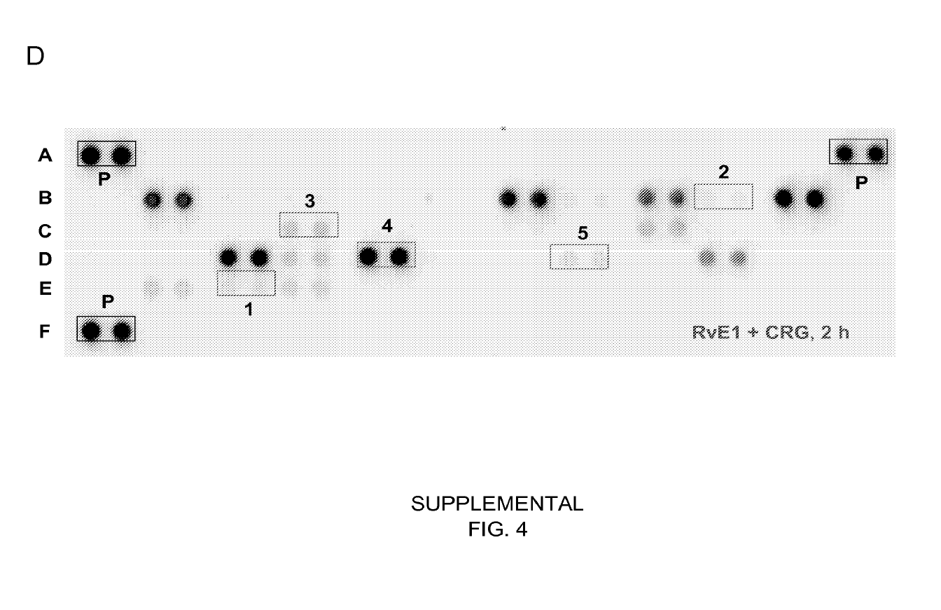

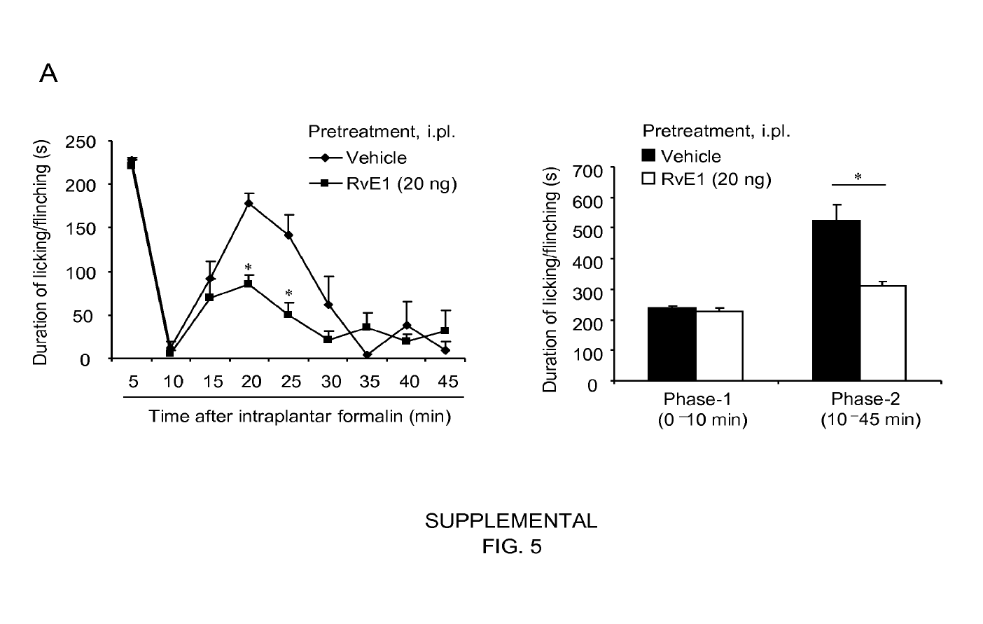

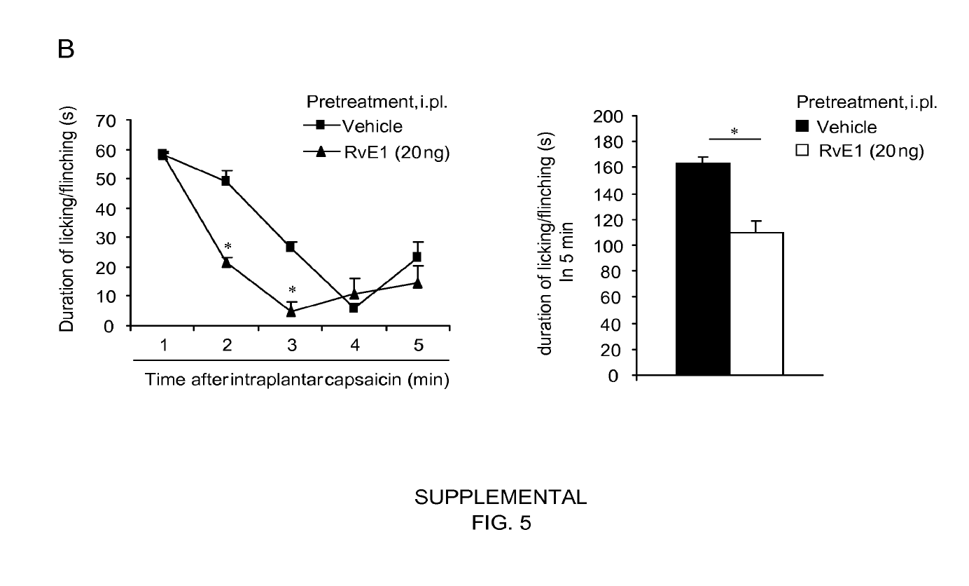

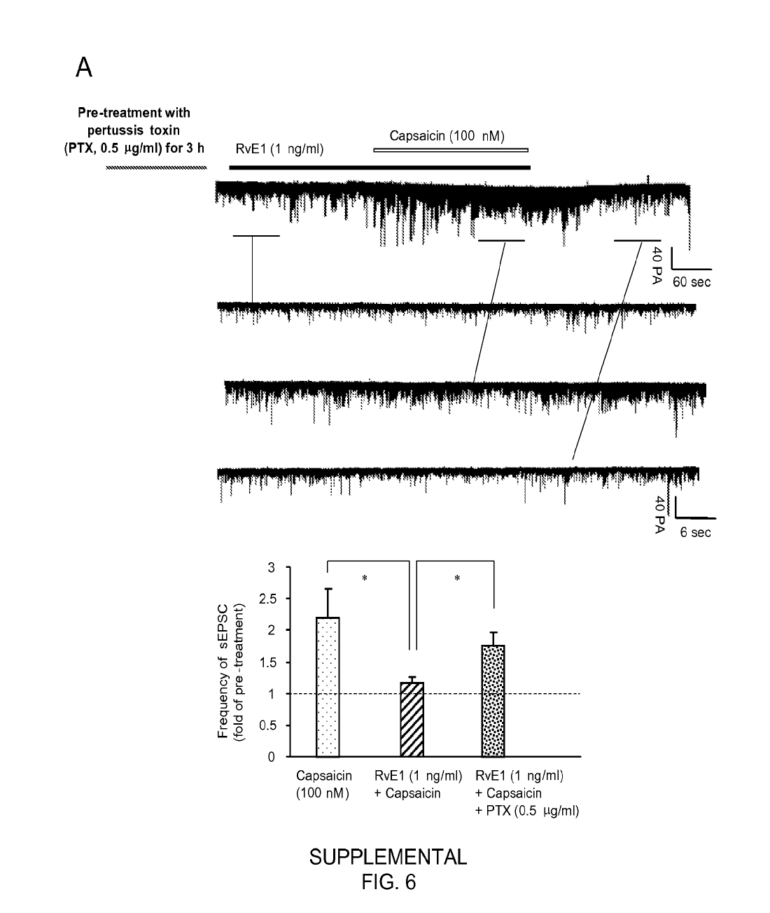

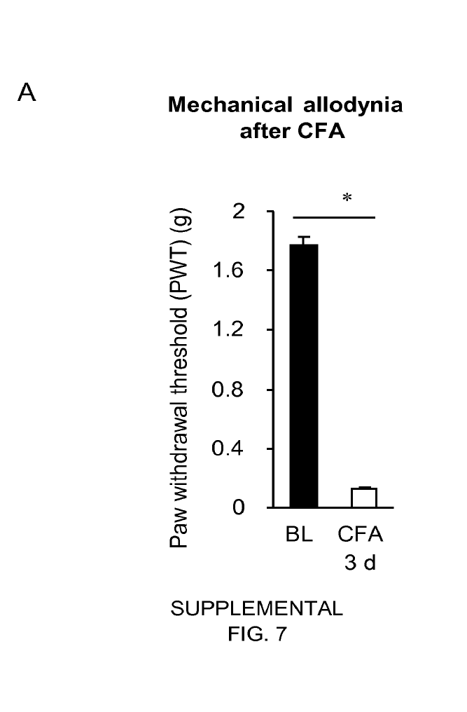

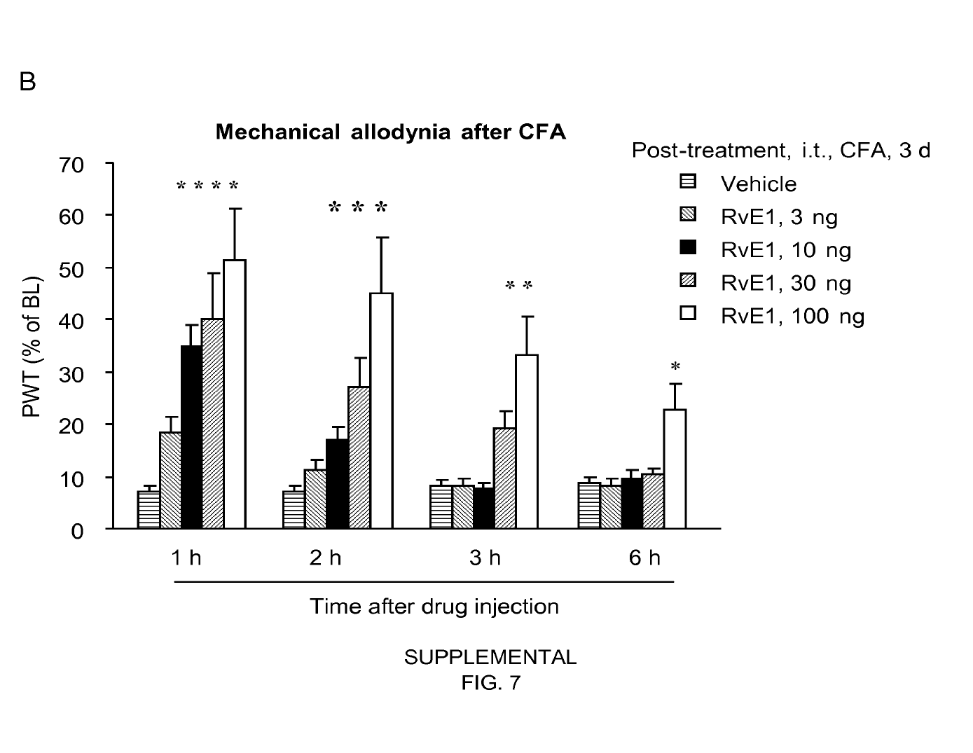

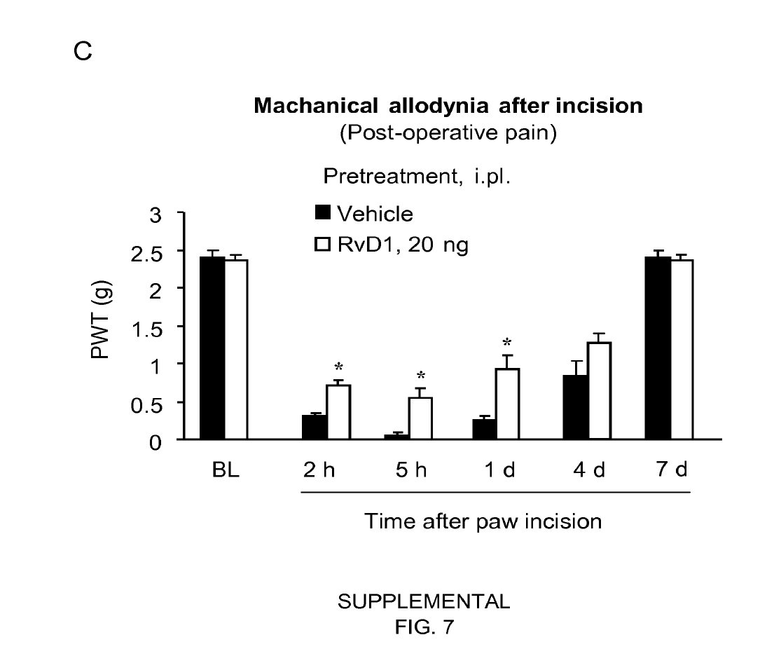

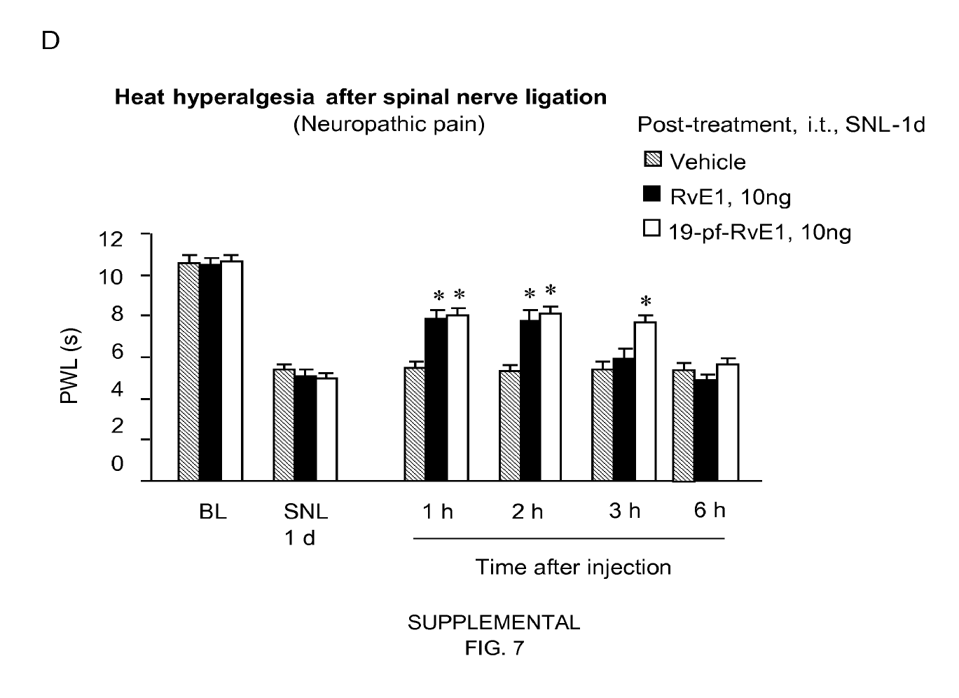

Supplementary FIG. 4. Cytokine array reveals a reduction of carrageenan (CRG)-induced expression of proinflammatory cytokines and chemokines in the inflamed hindpaw skin following intraplantar pre-treatment of RvE1. (a) List of all the proteins in the cytokine array. The array contains 40 different cytokines and chemokines with duplicates. The array also contains 3 positive control (PC) proteins. (b-d) Examples of three arrays (blots) that are incubated with skin tissue lysates from naïve (b), CRG-inflamed (c) and RvE1-treated inflamed (d) animals. Compared to naïve control, CRG inflammation for 2 h markedly increases the expression of multiple proinflammatory cytokines (e.g., TNF-α, IL-1β, and IL-6) and chemokines (e.g., MCP-1 and MIP-1α). Intraplantar administration of RvE1 (20 ng) prior to CRG injection inhibits CRG-induced expression of these cytokines and chemokines. All the arrays (blots) were processed under the same conditions, and this experiment was repeated 3 times in different animals. See FIG. 2h for the quantification.

Supplementary FIG. 5. Preemptive intraplantar (i.pl.) administration of RvE1 reduces formalin (a)- and capsaicin (b)-induced spontaneous pain. Left panels, time course of formalin (a)- or capsaicin (b)-induced nocifensive pain behavior. Right panels, summary of the data in the left panels. RvE1 was given 10 min before intraplantar formalin or capsaicin injection. Licking and flinching behaviors were assessed every 5 min after formalin injection and every minute after capsaicin injection. *P<0.05, compared to corresponding vehicle control, n=5.

Supplementary FIG. 6. RvE1 inhibits capsaicin-induced sEPSC increase in dorsal horn neurons via Gαi-associated GPCRs. Patch clamp recording in lamina II dorsal horn neurons shows that RvE1 fails to inhibit capsaicin-induced increases in sEPSC frequency after pretreatment with pertussix toxin (PTX). Low panel shows quantification of sEPSC frequency. Spinal cord slices were perfused with PTX (0.5 μg/ml) for 3 h before capsaicin stimulation. *P<0.05, n=5-9 neurons.

Supplementary FIG. 7. Resolvins attenuate mechanical allodynia or heat hyperalgesia in inflammatory, postoperative, and neuropathic pain conditions. (a,b) Intrathecal (i.t.) injection of RvE1 (3-100 ng), 3 d after CFA injection, reduces CFA-induced mechanical allodynia in a dose-dependent manner. a, development of mechanical allodynia at 3 d after CFA inflammation. PWT, paw withdrawal threshold. * P<0.05, compared to baseline (a) or vehicle (b). n=5-7. (c) Intraplantar (i.pl.) injection of RvD1 (20 ng), 10 min before paw incision, attenuates the development of paw incision-induced mechanical allodynia. *P<0.05, compared to corresponding vehicle control, n=5. (d) Intrathecal injection of RvE1 and modified RvE1 (10 ng, i.t.), one day after spinal nerve ligation (SNL), reduces SNL-induced heat hyperalgesia. Note that 19-pf-RvE1 produces a more persistent reduction in heat hyperalgesia. PWL, paw withdrawal latency; * P<0.05, compared to corresponding vehicle control. n=6.

### DETAILED DESCRIPTION

In the specification and in the claims, the terms “including” and “comprising” are open-ended terms and should be interpreted to mean “including, but not limited to . . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The term resolvin, as used herein, is recognized in the art and includes those compounds that are derived from DHA or EPA and are encompassed by U.S. patent application Ser. No. 09/785,866, filed Feb. 16, 2001, entitled “Aspirin Triggered Lipid Mediators” by Charles N. Serhan and Clary B. Clish, 10/639,714, filed Aug. 12, 2003, entitled “Resolvins: Biotemplates for Novel Therapeutic Interventions” by Charles N. Serhan and PCT Applications WO 01/60778, filed Feb. 16, 2001, entitled “Aspirin Triggered Lipid mediators” by Charles N. Serhan and Clary B. Clish and WO 04/014835, filed Aug. 12, 2003, entitled “Resolvins: Biotemplates for Novel Therapeutic Interventions” by Charles N. Serhan, non-naturally occurring structural analogs of trihydroxy polyunsaturated eicosanoids as described in US Publication 2003/0236423, U.S. Ser. No. 10/405,924, filed Apr. 1, 2003 and published Dec. 25, 2003 by Nicos Petasis, non-naturally occurring structural analogs of trihydroxy polyunsaturated eicosanoids as described in US Publication 2004/0044050, U.S. Ser. No. 10/460,913, filed Jun. 13, 2003 and published Mar. 4, 2004 by Daniel Goodman et al. and as described in the Journal of Biological Chemistry, 2007, vol. 282, number 13, pages 9323-9334, The Journal of Immunology, 2007; 178; 3912-3917, J. Am. Soc. Mass Spectrom. 2007 January; 18(1): 128-144, J. Immunol. 2008 Dec. 15; 181(12): 8677-8687 and J. Exp. Med. Volume 196, Number 8, Oct. 21, 2002 pages 1025-1037.

The present invention provides that in inflammatory pain models induced by formalin or complete Freund's adjuvant (CFA), resolvins such as RvE1, RvD1, PD1 potently inhibit pain-like behaviors. Notably, the doses required to suppress inflammatory pain is much lower than that of morphine and COX-2 inhibitor.

The present invention also provides that in a neuropathic pain model induced by spinal nerve ligation, resolvins such as RvE1 and PD1 also potently attenuate nerve injury-induced hyperalgesia.

The present invention further provides that in a postoperative pain model induced by surgical incision, resolvins such as RvE1 can effectively abolish the postoperative pain.

The RvE1 receptor ChemR23 is widely expressed in the nervous system. It is expressed in primary sensory neurons and secondary dorsal horn neurons, as well as in glial cells. Importantly, activation of ChemR23 by an endogenous ligand chemerin can effectively inhibit hyperalgesia.

Resolvins, such as RvE1 abolish, synaptic plasticity in the spinal cord dorsal horn neurons, induced by activation of nociceptive afferents.

Resolvins, such as RvE1 inhibit the activation of ERK/MAPK pathway in the spinal cord, an essential signaling pathway for the neural plasticity (sensitization of nociceptive neurons) in the spinal cord.

Resolvins, such as RvE1 blocks the TNF-α signaling pathway, a critical pathway for the pathogenesis of pain.

Inflammatory pain, such as arthritis pain, is a growing health problem¹. Inflammatory pain is generally treated with opioids and cyclooxygenase (COX) inhibitors, but both are limited by side effects. Recently, resolvins, a novel family of lipid mediators including RvE1 and RvD1 derived from omega-3 polyunsaturated fatty acid, show remarkable potency in treating disease conditions associated with inflammation^{2,3}. The present Mention provdies that peripheral (intraplantar) or spinal (intrathecal) administration of a resolvin, such as RvE1 or RvD1 (0.3-20 ng), potently reduces inflammatory pain behaviors in mice induced by intraplantar injection of formalin, carrageenan or complete Freund's adjuvant, without affecting basal pain perception. Intrathecal administration of a resolvin, such as RvE1, also inhibits spontaneous pain and heat and mechanical hypersensitivity evoked by intrathecal capsaicin and TNF-α. Resolvins, such as RvE1, play anti-inflammatory roles via reducing neutrophil infiltration, paw edema, and proinflammatory cytokine expression. Resolvins, such as RvE1′ also abolish TRPV1- and TNF-α-induced excitatory postsynaptic current increase and TNF-α-evoked NMDA receptor hyperactivity in spinal dorsal horn neurons, via inhibition of ERK signaling pathway. Thus, the present invention demonstrates a novel role of resolvins in normalizing spinal synaptic plasticity that has been implicated in generating pain hypersensitivity. Given the remarkable potency of resolvins and well known side effects of opioids and COX inhibitors, resolvins represent novel analgesics for treating inflammatory pain.

Resolution of acute inflammation, once thought to be a passive process, is now shown to involve active biochemical programs that enable inflamed tissues to return to homeostasis². The actions of pro-resolution mediators are in sharp contrast to those of currently used anti-inflammatory therapeutics. For example, inhibitors of COX and lipoxygenases disrupt resolution, because these enzymes are also required for the biosynthesis of pro-resolution mediators⁴⁻⁶. Resolvins, such as RvD1 and RvE1, are biosynthesized from omega-3 fatty acids docosahexaenoic acid (DHA) and eicosapentaenoic acid (EPA), respectively, and show remarkable potency in resolving inflammation-related diseases such as periodontal diseases, asthma, and retinopathy^{2,3,7}. Peripheral and central mechanisms of inflammatory pain are not fully understood⁸⁻¹¹. The present invention examined whether peripheral and central resolvins can attenuate inflammatory pain, and further how resolvins regulate synaptic plasticity in spinal cord dorsal horn neurons that has been strongly implicated in the generation of persistent pain^{10,11}.

The actions of resolvins, such as RvE1, were examined in an acute inflammatory pain condition induced by intraplantar injection of formalin. Formalin induced characteristic two-phase spontaneous pain behavior, and the second phase is likely mediated by spinal cord mechanisms^{12,13}. Synthetic resolvins were delivered to the mouse spinal cord via intrathecal (i.t.) route using lumbar puncture^{14,15}. Surprisingly, preemptive injection of a resolvin, such as RvE1, at very low doses, only 0.3 and 1.0 ng (i.e. 1 and 3 pmol), reduced the 2^{nd} but not the 1^{st} phase pain behavior, suggesting a possible central action of resolvins, e.g., RvE1 (FIG. 1a, b). Notably, the effective dose range of RvE1 was much lower than that of either morphine or the COX-2 inhibitor NS-398 (FIG. 1c).

Resolvin were further investigated where RvE1's antinociceptive action was mediated by specific receptors. ChemR23, which is associated with G-protein subunit Gai, was identified as RvE1's receptor^{16,17}. Spinal injection of Gai inhibitor pertussis toxin (PTX) abrogated resolvin, RvE1's, action (FIG. 1d), suggesting a possible involvement of GPCRs. Opioid receptors did not mediate, for example, RvE1's antinociceptive action, as opioid receptor antagonist naloxone reversed morphine but not RvE1's effect (FIG. 1d). Chemerin, a peptide agonist for ChemR23¹⁸, also dose-dependently attenuated formalin-induced 2^{nd} phase pain (FIG. 1e). Notably, knockdown of ChemR23 with a specific siRNA abolished RvE1's antinociceptive actions (Supplementary FIG. 1). In situ hybridization revealed an expression of ChemR23 mRNA in the dorsal root ganglion (DRG) and spinal cord (FIG. 1f). Double staining further demonstrated an expression of ChemR23 protein in DRG neurons that co-express transient potential receptor vanilloid subtype-1 (TRPV1) (FIG. 1g; Supplementary FIG. 2) and in spinal cord cells that co-express the neuronal marker NeuN (Supplementary FIG. 3a-c). It was also found that ChemR23 in axons of DRG neurons (FIG. 1g) and primary afferent terminals in the spinal cord (Supplementary FIG. 3d). Therefore, RvE1 might attenuate inflammatory pain via ChemR23 expressed in DRG and spinal cord neurons.

Intraplantar injection of complete Freund's adjuvant (CFA) elicits persistent inflammatory pain for weeks¹⁹. Intrathecal resolvins, given on post-CFA day 3 when heat hyperalgesia (reduction of paw withdrawal latency) was fully developed (FIG. 2a), attenuated this hyperalgesia in a dose-dependently manner (FIG. 2b,c). Notably, 10 ng RvE1 produced ˜75% reduction in hyperalgesia at 15 min after administration (FIG. 2b). A meta-analysis demonstrates that dietary omega-3 fatty acids alleviate inflammatory pain in patients²⁰. The omega-3 fatty acids EPA and DHA, the respective precursors of resolvins such as RvE1 and RvD1, also reduced CFA-evoked heat hyperalgesia. But the effective doses of EPA and DHA required were 10,000 times higher than that of resolvins such as RvE1 (FIG. 2b). For direct comparison, 10 ng RvE1 was more potent than 10 μg COX-2 inhibitor NS-398 (FIG. 2c). Notably, an RvE1 stable analog, 19-(p-fluorophenoxy)-RvE1 (19-pf-RvE1), designed to resist rapid local metabolic inactivation of RvE1²¹, reduced hyperalgesia for 6 h (FIG. 2c). By contrast, the further metabolic product of RvE1 namely 18-oxo-RvE1²¹ was essentially inactive (FIG. 2c). Although RvE1 potently reduced inflammatory pain, it did not alter baseline sensory thresholds in naïve mice (FIG. 2d). These findings suggest that resolvins play a unique role in “normalization” of inflammatory pain.

We further investigated the peripheral role of resolvins in carrageenan (CRG)-elicited pain and inflammation. Intraplantar pretreatment of a resolvin, such as RvD1 or RvE1, substantially attenuated CRG-evoked heat hyperalgesia (FIG. 2e). As expected, resolvins, such as RvE1, also generated marked anti-inflammatory actions, by reducing CRG-induced edema, neutrophil infiltration, and expression of proinflammatory cytokines (e.g., TNF-α, IL-1β, IL-6) and chemokines (e.g., MCP-1, MIP-1α) in inflamed hindpaws (FIG. 2f-h; Supplementary FIG. 4). Intraplantar RvE1 also rapidly attenuated formalin-evoked acute pain (Supplementary FIG. 5a).

To determine potential mechanisms by which resolvin attenuates inflammatory pain, the impact of a resolvin, such as RvE1, on TNF-α signaling was examined, because TNF-α contributes importantly to the genesis of inflammation and pain via both peripheral^{22,23} and central mechanisms²⁴. Indeed, Tnfr^{-/-} mice displayed a marked attenuation in CFA-elicited heat hyperalgesia and formalin-elicited 2^{nd} phase pain (FIG. 3a). Intrathecal injection of TNF-α also evoked marked heat hyperalgesia, which was abrogated in mice lacking Trpv1, a critical gene for generating heat hyperalgesia²⁵. In contrast, formalin-induced 2″-phase spontaneous pain was unaltered in Trpv1^{-/-} mice (FIG. 3b). Spinal administration of a resolvin, such as RvE1, substantially reduced TNF-α-induced heat hyperalgesia (FIG. 3c). Hence, resolvins, such as RvE1, can alleviate both TRPV1-dependent and independent inflammatory pain symptoms.

To determine whether resolvins modulate spinal cord synaptic plasticity underlying the generation of inflammatory pain^{10,11}, a patch clamp technique was used to record spontaneous excitatory postsynaptic currents (sEPSCs) in lamina II neurons ex vivo in isolated spinal cord slices. Perfusion of spinal cord slice with TNF-α induced an increase in the frequency but not amplitude of sEPSCs (FIG. 3d), suggesting a presynaptic effect of TNF-α by increasing glutamate release from axonal terminals²⁴. Notably, resolvins such as RvE1, alone did not alter basal synaptic transmission but blocked TNF-α-evoked sEPSC frequency increase (FIG. 3d). The TRPV1 antagonist capsazepine also reduced this frequency increase by TNF-α (FIG. 3d), in parallel with earlier results that TNF-α increased TRPV1 activity in DRG neurons^{26,27}. Direct activation of TRPV1 by capsaicin (100 nM) elicited a 2-fold increase in sEPSC frequency, which was completely blocked by resolvins, e.g., RvE1. Like RvE1, chemerin also abolished this frequency increase by capsaicin, in a PTX-dependent manner, indicating an involvement of ChemR23 (FIG. 3e; Supplementary FIG. 6). Notably, intrathecal capsaicin elicited acute spontaneous pain for <10 min, and intrathecal administration of a resolvin, e.g., RvE1, prevented this pain (FIG. 3f). In parallel, peripheral administration of a resolvin, such as RvE1, reduced intraplantar capsaicin-induced acute pain (Supplementary FIG. 5b). These results further established that resolvins, such as RvE1, attenuate inflammatory pain by blocking TRPV1 and TNF-α signaling, presumably at presynaptic sites.

Next, it eas investigated whether resolvins regulate synaptic plasticity via the extracellular signal-regulated kinase (ERK) signaling pathway, because previous studies reported that (1) ChemR23 regulates ERK signaling in non-neuronal cells¹⁸, (2) ERK activation in DRG neurons increases TRPV1 activity²⁸, and (3) ERK modulates neurotransmitter release via phosphorylation of synapsin I²⁹. The ERK pathway was inhibited with MEK inhibitor U0126 or PD98059 and it was found that both blocked capsaicin-induced sEPSC increase, indicating an essential role of ERK in regulating presynaptic glutamate release in the spinal cord (FIG. 3e). In dissociated DRG neurons, both TNF-α and capsaicin elicited increases in phosphorylation of ERK (pERK) and resolvins, such as RvE1, abolished these increases (FIG. 3g). Thus, resolvins such as RvE1, might attenuate inflammatory pain by blocking ERK-mediated glutamate release in presynaptic terminals, in response to TNF-α stimulation and TRPV1 activation (FIG. 3h).

Apart from heat hyperalgesia, CFA and intrathecal TNF-α produced another cardinal feature of inflammatory pain, mechanical allodynia, a reduction in paw withdrawal threshold. Intrathecal administration of a resolvin such as RvE1 also attenuated mechanical allodynia induced by TNF-α or CFA (FIG. 4a; Supplementary FIG. 7a,b). Of note TNF-α-elicited mechanical allodynia was TRPV1-independent (FIG. 4b). To define potential mechanisms by which resolvins, such as RvE1, attenuate mechanical allodynia, the activation of glutamate NMDA receptors (NMDARs) was examined in dorsal horn neurons, which result in hyperactivity of these neurons (central sensitization) and subsequent mechanical allodynia^{10,19}. The activity of NMDARs was measured by recording NMDA-induced currents in dorsal horn neurons. TNF-α significantly potentiated NMDA currents, and resolvins, such as RvE1, blocked this potentiation (FIG. 4c). It ws further assessed whether RvE1 inhibits NMDAR activation via the ERK pathway, as ERK phosphorylation in dorsal horn neurons serves as a marker for central sensitization^{13,30}. Perfusion of spinal slices with TNF-α induced a robust ERK phosphorylation primarily in superficial dorsal horn neurons, and resolvin RvE1 treatment reduced the phosphorylation (FIG. 4d). ERK mediates central sensitization via activation of NMDARs in postsynaptic dorsal horn neurons, because the MEK inhibitor PD98059 and U0126 but not capsazepine blocked TNF-α-induced NMDAR activation (FIG. 4e). Thus, resolvins such as RvE1 also attenuate the inflammatory pain by inhibiting ERK-mediated NMDAR activation in postsynaptic dorsal horn neurons (FIG. 4f).

In addition to dampening behavioral hypersensitivity in inflammatory pain conditions, resolvins also reduced mechanical or heat hypersensitivity in other persistent pain conditions, such as incision-induced postoperative pain (Supplementary FIG. 7c) and nerve injury-induced neuropathic pain (Supplementary FIG. 7d).

In summation, these results demonstrated that resolvins, at very low doses (about 0.1 to about 20 ng), effectively reduced inflammatory pain symptoms in several mouse models, via both peripheral and central actions. Biosynthesized during resolution of acute inflammation, resolvins are known to act on immune cells to produce anti-inflammatory actions (e.g., reducing polymorphonuclear leukocyte infiltration and tissue injury) and pro-resolving actions (e.g., increasing phagocytosis activity of macrophages)². Peripheral administration of resolvins, such as RvE1, reduced CRG-elicited expression of proinflammatory cytokines, neutrophil infiltraton, and paw edema. Since the proinflammatory cytokines such as TNF-α and IL-1β are indispensable for the pathogenesis of inflammatory pain (FIG. 3a, b)^{24,31}, resolvin's antinociceptive actions could be attributable to its anti-inflammatory role. Hence it is particularly noteworthy that it was demonstrated herein a novel mechanism in pain resolution in which resolvins, such as RvE1, rapidly, within minutes, reduced inflammatory pain via modulating synaptic plasticity in dorsal horn neurons (FIG. 3h, 4f). Resolvins, such as RvE1, not only abolished TRPV1-induced EPSC frequency increase and spontaneous pain, but also blocked TNF-α-induced EPSC frequency increase and NMDAR hyperactivity. As illustrated in FIG. 3h and FIG. 4f, RvE1 requires the activation of the GPCR ChemR23 and the inactivation of the ERK signaling pathway in both presynaptic and postsynaptic neurons for mediating its antinociceptive actions.

Current treatments for inflammatory pain are limited by side effects, such as respiratory depression, sedation, nausea, vomiting, constipation, dependence, tolerance, and addiction after opioid treatment^{32,33} and serious cardiovascular effects associated with long-term treatment of COX-2 inhibitors^{1,34}. Also, COX-2 inhibitors and local anesthetic impair the resolution of acute inflammation^{4,6}. Although enthusiasm for TRPV1 antagonists is high, these drugs can cause hyperthermia³⁵ and have limited efficacy on mechanical allodynia. The present results show that resolvins are potent in attenuating inflammatory pain without changing basal pain sensitivity. Given the remarkable anti-hyperalgesic efficacy of resolvins and safety associated with endogenous mediators, resolvins and their metabolically stable analogues may represent a novel family of analgesics useful for treating inflammation-associated pain such as arthritic pain and postoperative pain. This new analgesic function gives a unique feature that now adds to the beneficial anti-inflammatory and pro-resolving actions of resolvins².

Compounds Useful in the Invention

The present invention, in one embodiment, is drawn to uses described throughout the specification with isolated therapeutic agents generated from the interaction between a dietary omega-3 polyunsaturated fatty acid (PUFA) such as eicosapentaenoic acid (EPA) or docosahexaenoic acid (DHA), an oxygenase, such as cyclooxygenase-II (COX-2), and an analgesic, such as aspirin (ASA). Surprisingly, careful and challenging isolation of previously unknown and unappreciated compounds are generated from exudates by the combination of components in an appropriate environment to provide di- and tri-hydroxy EPA and DHA derivatives having unique structural and physiological properties. The present invention therefore provides for many new useful therapeutic di- and tri-hydroxy derivatives of EPA or DHA that diminish, prevent, or eliminate the disorders, conditions and/or diseases described herein.

Resolvins, such as resolvin E1 (RvE1; 5S,12R,18R-trihydroxyeicosapentaenoic acid) are novel anti-inflammatory lipid mediators derived from omega-3 fatty acid eicosapentaenoic acid (EPA).

The di- and tri-hydroxy EPA and DHA therapeutic agents of the invention useful to treat the disorders, conditions and/or diseases include, for example:

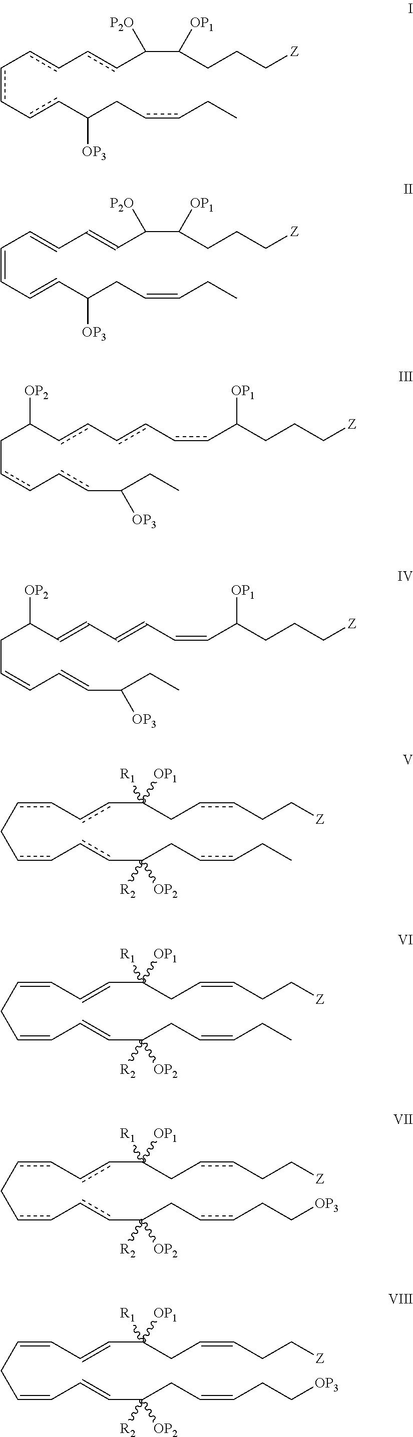

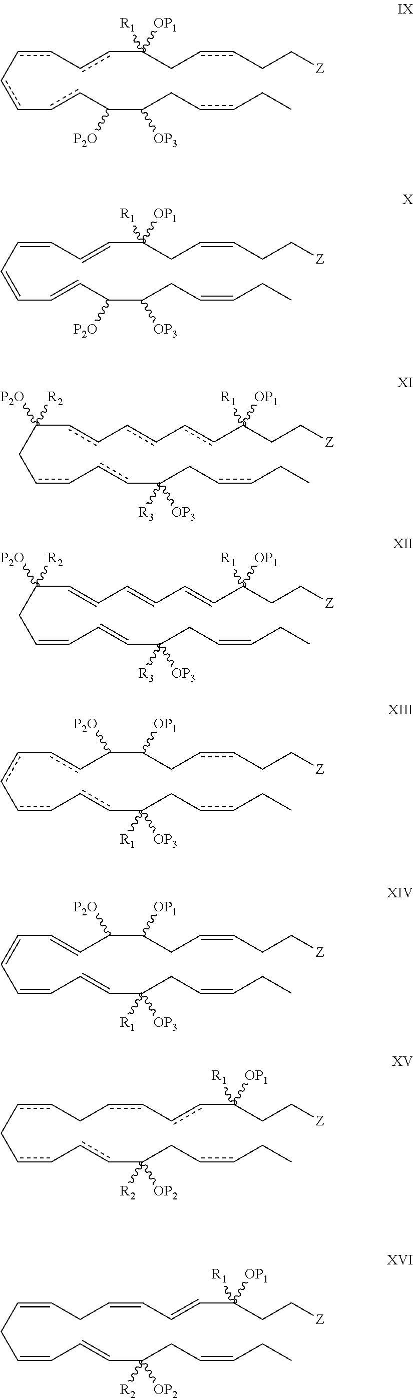

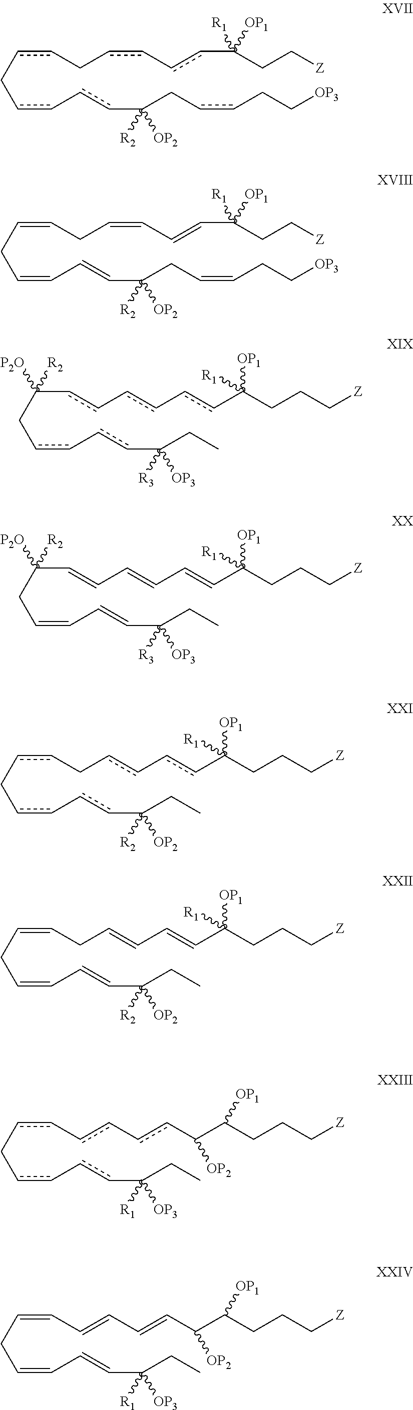

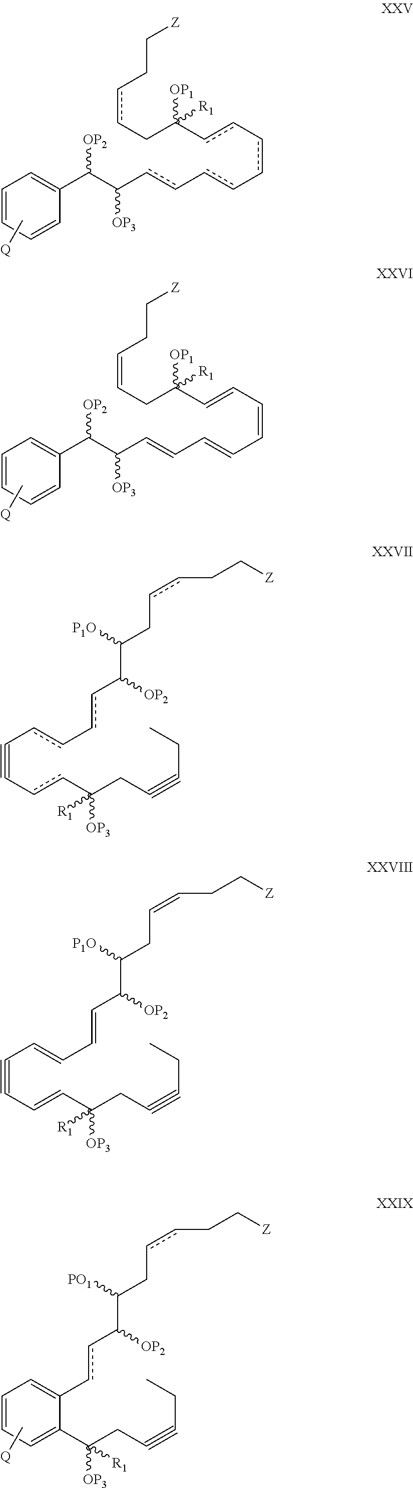

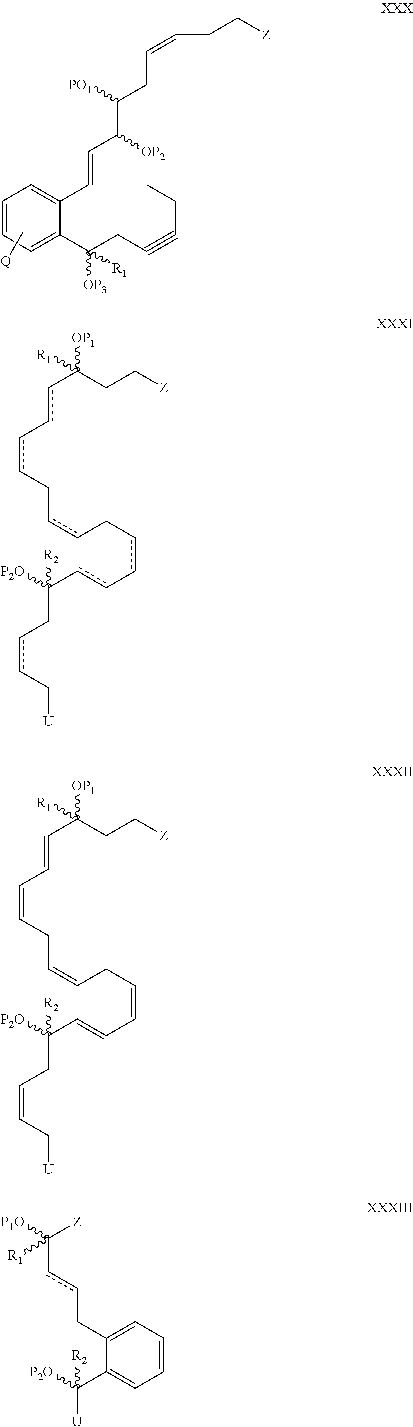

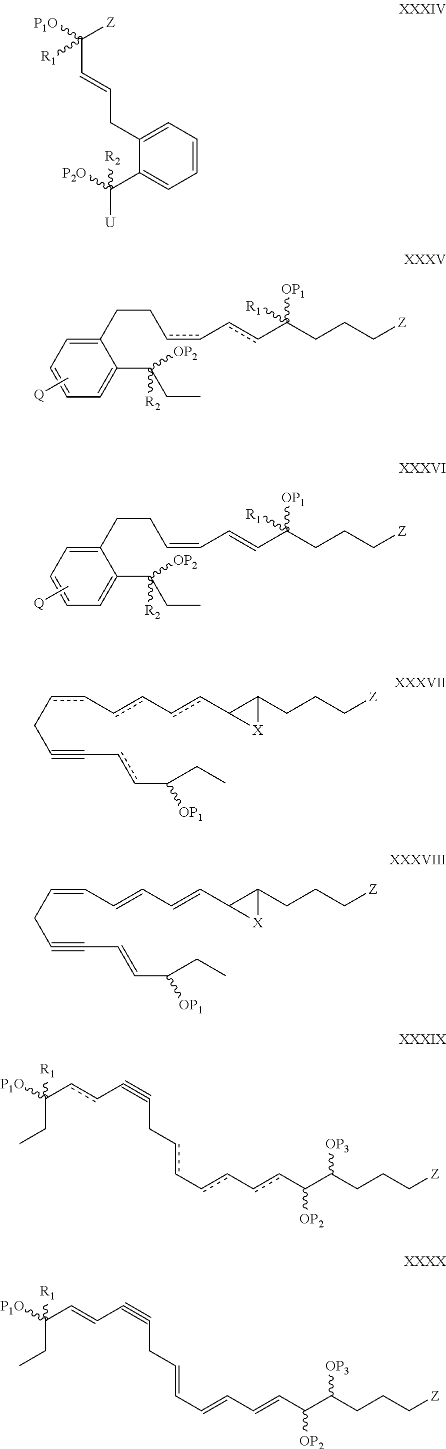

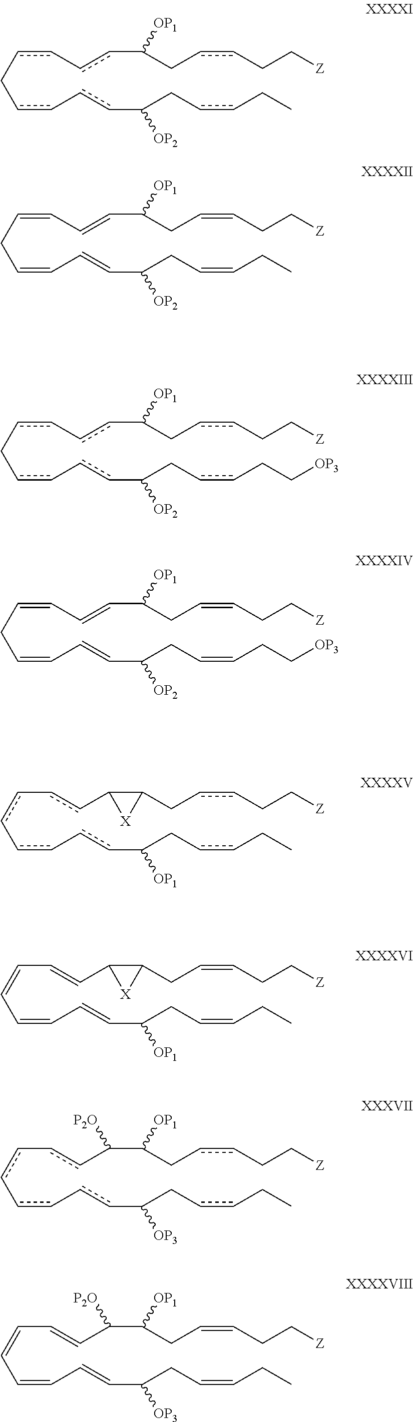

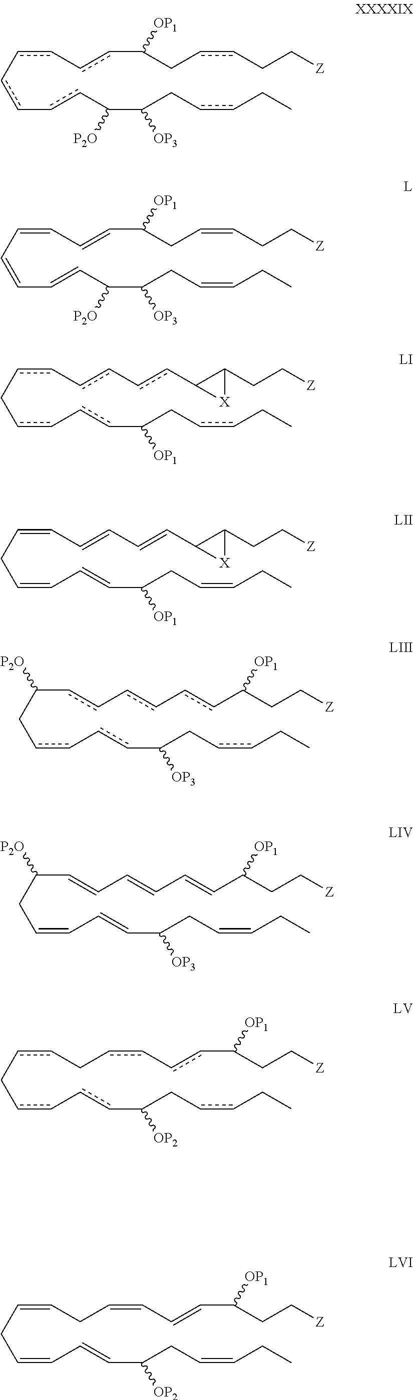

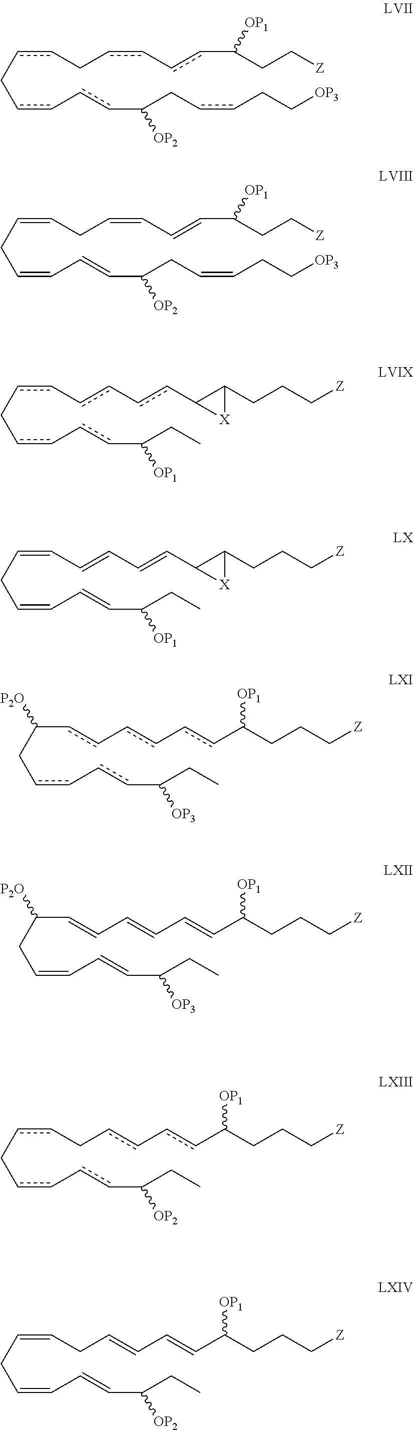

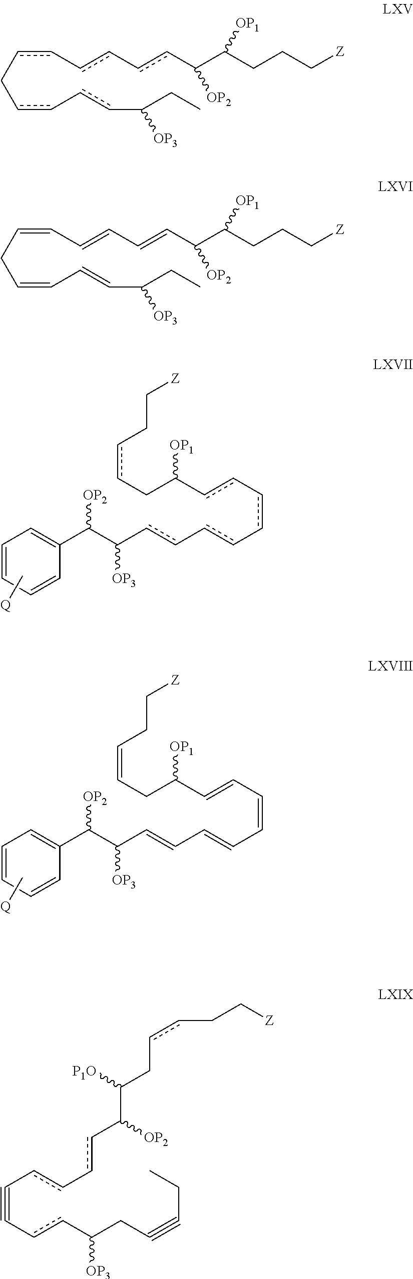

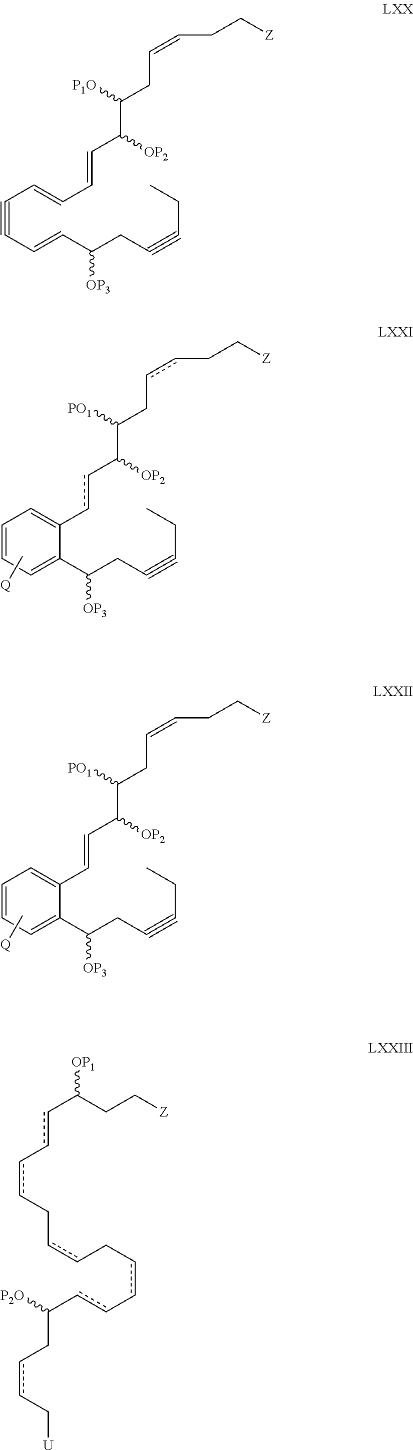

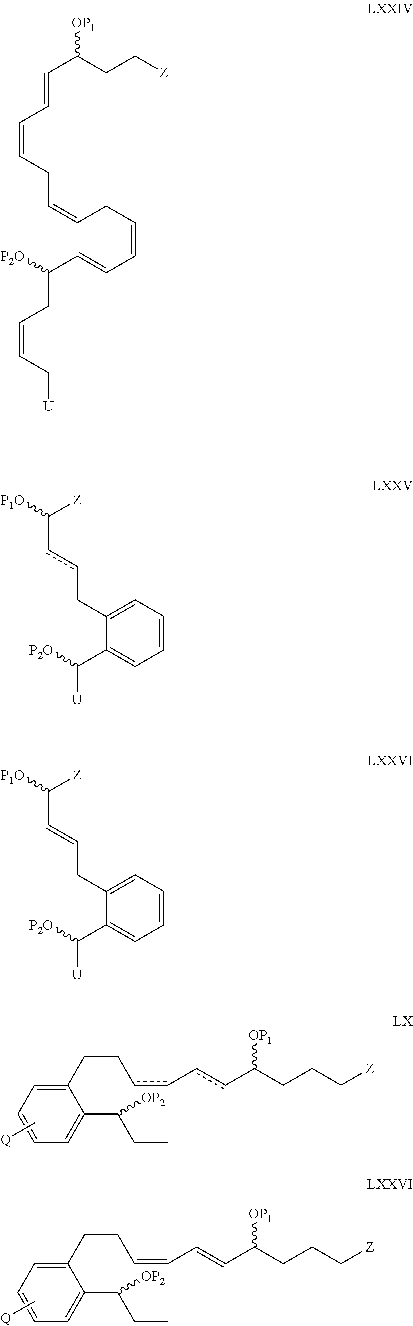

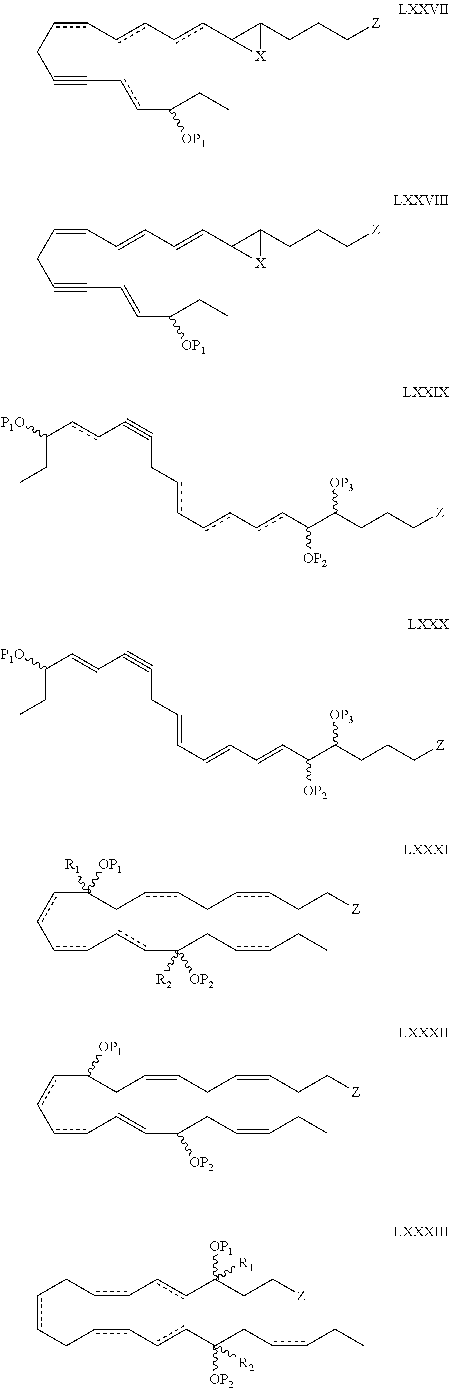

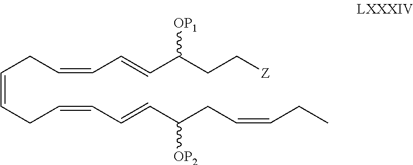

wherein a bond depicted as [symbol] represents either a cis or trans double bond;

wherein P₁, P₂ and P₃, if present, each individually are protecting groups, hydrogen atoms or combinations thereof;

wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;

wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;

each R^{a}, if present, is independently selected from the group consisting of hydrogen, (C1-C6) alkyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b}, if present, is a suitable group independently selected from the group consisting of ═O, —OR^{d}, (C1-C3)haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;

each n, independently, if present, is an integer from 0 to 3;

each R^{d}, independently, if present, is a protecting group or R^{a};

in particular, Z is a carboxylic acid, ester, amide, thiocarbamate, carbamate, thioester, thiocarboxamide or a nitrile;

wherein X, if present, is a substituted or unsubstituted methylene, an oxygen atom, a substituted or unsubstituted nitrogen atom, or a sulfur atom;

wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;

wherein U, if present, is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;

and pharmaceutically acceptable salts thereof.

In certain embodiments, Z is a pharmaceutically acceptable salt of a carboxylic acid, and in particular is an ammonium salt or forms a prodrug.

In certain embodiments, P₁, P₂, and P₃, if present, each individually are hydrogen atoms and Z is a carboxylic acid or ester. In other embodiments, X is an oxygen atom, one or more P's are hydrogen atoms, and Z is a carboxylic acid or ester. In still other embodiments, Q is one or more halogen atoms, one or more P's are hydrogen atoms, and Z is a carboxylic acid or ester.

In certain embodiments, R₁, R₂ and R₃, if present, are each individually lower alkyl groups, such as methyl, ethyl, and propyl and can be halogenated, such as trifluoromethyl. In one aspect, at least one of R₁, R₂ and R₃, if present, is not a hydrogen atom. Generally, Z is a carboxylic acid and one or more P's are hydrogen atoms.

In certain embodiments, when OP₃ is disposed terminally within the resolvin analog, the protecting group can be removed to afford a hydroxyl. Alternatively, in certain embodiments, the designation of OP₃ serves to denote that the terminal carbon is substituted with one or more halogens, i.e., the terminal C-18, C-20, or C-22 carbon, is a trifluoromethyl group, or arylated with an aryl group that can be substituted or unsubstituted as described herein. Such manipulation at the terminal carbon serves to protect the resolvin analog from omega P₄₅₀ metabolism that can lead to biochemical inactivation.

In certain embodiments, P₁, P₂, and P₃, if present, each individually are hydrogen atoms and Z is a carboxylic ester. In other embodiments, P₁, P₂, and P₃, if present, each individually are hydrogen atoms and Z is not carboxylic acid.

In one aspect, the compounds described herein are isolated and/or purified, in particular, compounds in which P₁, P₂, and P₃, if present, each individually are hydrogen atoms and Z is a carboxylic acid, are isolated and or purified.

In one aspect, the resolvins described herein that contain epoxide, cyclopropane, azine, or thioazine rings within the structure also serve as enzyme inhibitors that increase endogenous resolvin levels in vivo and block “pro” inflammatory substances, their formation and action in vivo, such as leukotrienes and/or LTB₄.

Another embodiment of the present invention is directed to pharmaceutical compositions of the novel compounds described throughout the specification useful to treat the conditions described herein.

The present invention also provides methods to treat the disease states and conditions described herein.

The present invention also provides packaged pharmaceuticals that contain the novel di- and tri-hydroxy EPA and DHA derivatives described throughout the specification for use in treatment with the disease states and conditions described herein.

It should be understood that throughout the specification, all compounds, including intermediates, can be isolated and purified by methods known in the art, such as distillation, chromatography, crystallization, filtration, HPLC, etc. The purity of the compound can be from about 80% to about 100%, in particular from about 85% to about 99.9%, more particularly from about 90% to about 99.5% and even more particularly from about 95% to about 99.9%.

“Alkyl” by itself or as part of another substituent refers to a saturated or unsaturated branched, straight-chain or cyclic monovalent hydrocarbon radical having the stated number of carbon atoms (i.e., C₁-C₆ means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. In preferred embodiments, the alkyl groups are (C₁-C₆) alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In preferred embodiments, the alkanyl groups are (C1-C6) alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C2-C6) alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C2-C6) alkynyl.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group is (C1-C6) alkyldiyl. Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3) alkyleno. Also preferred are straight-chain saturated alkano groups, e.g., methano, ethano, propano, butano, and the like.

“Heteroalkyl,” Heteroalkanyl,” Heteroalkenyl,” Heteroalkynyl,” Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O) NR′—, —S(O)₂NR′—, and the like, including combinations thereof, where each R′ is independently hydrogen or (C1-C6) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C5-C15) aryl, with (C5-C10) being even more preferred. Particularly preferred aryls are cyclopentadienyl, phenyl and naphthyl.

“Arylaryl” by itself or as part of another substituent refers to a monovalent hydrocarbon group derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. Where the number of carbon atoms in an arylaryl group are specified, the numbers refer to the carbon atoms comprising each parent aromatic ring. For example, (C5-C15) arylaryl is an arylaryl group in which each aromatic ring comprises from 5 to 15 carbons, e.g., biphenyl, triphenyl, binaphthyl, phenylnaphthyl, etc. Preferably, each parent aromatic ring system of an arylaryl group is independently a (C5-C15) aromatic, more preferably a (C5-C10) aromatic. Also preferred are arylaryl groups in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. Preferably, the aromatic ring systems are (C5-C15) aromatic rings, more preferably (C5-C10) aromatic rings. A particularly preferred biaryl group is biphenyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In preferred embodiments, the arylalkyl group is (C6-C21) arylalkyl, e.g., the alkanyl, alkenyl or alkynyl moiety of the arylalkyl group is (C1-C6) and the aryl moiety is (C5-C15). In particularly preferred embodiments the arylalkyl group is (C6-C13), e.g., the alkanyl, alkenyl or alkynyl moiety of the arylalkyl group is (C1-C3) and the aryl moiety is (C5-C10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

“Heteroaryl-Heteroaryl” by itself or as part of another substituent refers to a monovalent heteroaromatic group derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. Where the number of atoms are specified, the numbers refer to the number of atoms comprising each parent heteroaromatic ring systems. For example, 5-15 membered heteroaryl-heteroaryl is a heteroaryl-heteroaryl group in which each parent heteroaromatic ring system comprises from 5 to 15 atoms, e.g., bipyridyl, tripuridyl, etc. Preferably, each parent heteroaromatic ring system is independently a 5-15 membered heteroaromatic, more preferably a 5-10 membered heteroaromatic. Also preferred are heteroaryl-heteroaryl groups in which all of the parent heteroaromatic ring systems are identical.

“Biheteroaryl” by itself or as part of another substituent refers to a heteroaryl-heteroaryl group having two identical parent heteroaromatic ring systems joined directly together by a single bond. Typical biheteroaryl groups include, but are not limited to, bipyridyl, bipurinyl, biquinolinyl, and the like. Preferably, the heteroaromatic ring systems are 5-15 membered heteroaromatic rings, more preferably 5-10 membered heteroaromatic rings.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR′″, where R′″ is a haloalkyl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

Throughout the following descriptions, it should be understood that where particular double bonding is depicted, it is intended to include both cis and trans configurations. Exemplary formulae are provided with specific configurations, but for completeness, the double bonds can be varied. Not every structural isomer is shown in efforts to maintain brevity of the specification. However, this should not be considered limiting in nature. Additionally, where synthetic schemes are provided, it should be understood that all cis/trans configurational isomers are also contemplated and are within the scope and purvue of the synthesis. Again, particular double bonding is depicted in exemplary manner.

In one embodiment, the analogs are designated as 10, 17-diHDHAs. P₁ and P₂ are as defined above and can be the same or different. Z is as defined above and in particular can be a carboxylic acid, ester, amide, thiocarbamate, carbamate, thioester, thiocarboxamide or a nitrile. The broken double bond line, where noted, indicates that either the E or Z isomer is within the scope of the analog(s). In certain aspects, the chiral carbon atom at the 10 position (C-10) has an R configuration. In another aspect, the C-10 carbon atom has an S configuration. In still another aspect, the C-10 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate. In one example, the present invention includes 10,17S-docosatriene, 10,17S— dihydroxy-docosa-4Z,7Z,11E,13,15E,19Z-hexaenoic acid analogs such as 10R/S—OCH₃,17S-HDHA, 10R/S, methoxy-17S hydroxy-docosa-4Z,7Z,11E,13,15E,19Z-hexaenoic acid derivatives.

In certain embodiments, when P₁ and P₂ are hydrogen atoms and Z is a carboxylic acid, the compound is either isolated and/or purified.

In still yet another embodiment, the present invention pertains to diHDHA analogs that are designated as 4,17-diHDHAs. P₁, P₂ and Z are as defined above. P₁ and P₂ can be the same or different. In particular, Z can be a carboxylic acid, ester, amide, thiocarbamate, carbamate, thioester, thiocarboxamide or a nitrile. In certain aspects, the chiral carbon atom at the 4 position (C-4) has an R configuration. In another aspect, the C-4 carbon atom preferably has an S configuration. In still another aspect, the C-4 carbon atom is as an R/S racemate. Additionally, the chiral carbon atom at the 17 position (C-17) can have an R configuration. Alternatively, the C-17 carbon can have an S configuration. In still yet another aspect, the C-17 carbon can preferably exist as an R/S racemate.

In certain embodiments, when P₁ and P₂ are hydrogen atoms and Z is a carboxylic acid, the compound is either isolated and/or purified.

For example, the present invention includes 4S, 17R/S-diHDHA, 4S,17R/S-dihydroxy-docosa-5E,7Z,10Z,13Z,15E,19Z-hexaenoic acid analogs.

It should be understood that “Z” can be altered from one particular moiety to another by a skilled artisan. In order to accomplish this in some particular instances, one or more groups may require protection. This is also within the skill of an ordinary artisan. For example, a carboxylic ester (Z) can be converted to an amide by treatment with an amine. Such interconversions are known in the art.

In the compounds described herein, it should be understood that reference to “hydroxyl” stereochemistry is exemplary, and that the term is meant to include protected hydroxyl groups as well as the free hydroxyl group. In certain embodiments, the C-17 position has an R configuration. In other embodiment, the C-17 position has an S configuration. In other aspects, certain embodiments of the invention have an R configuration at the C-18 position.

In certain aspects of the present invention, ASA pathways generate R>S and therefore, 4S, 5R/S, 7S,8R/S, 11R,12 R/S16 S, 17 R. With respect to species generated from the 15-LO pathway the chirality of C-17 is S , C-16 R and C-10, preferably R.

The hydroxyl(s) in the compounds described hereincan be protected by various protecting groups (P), such as those known in the art. An artisan skilled in the art can readily determine which protecting group(s) may be useful for the protection of the hydroxyl group(s). Standard methods are known in the art and are more fully described in literature. For example, suitable protecting groups can be selected by the skilled artisan and are described in Green and Wuts, “Protecting Groups in Organic Synthesis”, John Wiley and Sons, Chapters 5 and 7, 1991, the teachings of which are incorporated herein by reference. Preferred protecting groups include methyl and ethyl ethers, TMS or TIPPS groups, acetate (esters) or propionate groups and glycol ethers, such as ethylene glycol and propylene glycol derivatives.

For example, one or more hydroxyl groups can be treated with a mild base, such as triethylamine in the presence of an acid chloride or silyl chloride to facilitate a reaction between the hydroxyl ion and the halide. Alternatively, an alkyl halide can be reacted with the hydroxyl ion (generated by a base such as lithium diisopropyl amide) to facilitate ether formation.

The compounds can be prepared by methods provided in U.S. patent application Ser. No. 09/785,866, filed Feb. 16, 2001, entitled “Aspirin Triggered Lipid Mediators” by Charles N. Serhan and Clary B. Clish, 10/639,714, filed Aug. 12, 2003, entitled “Resolvins: Biotemplates for Novel Therapeutic Interventions” by Charles N. Serhan and PCT Applications WO 01/60778, filed Feb. 16, 2001, entitled “Aspirin Triggered Lipid mediators” by Charles N. Serhan and Clary B. Clish and WO 04/014835, filed Aug. 12, 2003, entitled “Resolvins: Biotemplates for Novel Therapeutic Interventions” by Charles N. Serhan, the contents of which are incorporated herein by reference in their entirety.

It should also be understood that for the compounds described herein, not all hydroxyl groups need be protected. One, two or all three hydroxyl groups can be protected. This can be accomplished by the stoichiometric choice of reagents used to protect the hydroxyl groups. Methods known in the art can be used to separate the di- or tri-protected hydroxy compounds, e.g., HPLC, LC, flash chromatography, gel permeation chromatography, crystallization, distillation, etc.

It should be understood that there are one or more chiral centers in each of the above-identified compounds. It should understood that the present invention encompasses all stereochemical forms, e.g., enantiomers, diastereomers and racemates of each compound. Where asymmetric carbon atoms are present, more than one stereoisomer is possible, and all possible isomeric forms are intended to be included within the structural representations shown. Optically active (R) and (S) isomers may be resolved using conventional techniques known to the ordinarily skilled artisan. The present invention is intended to include the possible diastereiomers as well as the racemic and optically resolved isomers.

The resolvin analogs depicted throughout the specification contain acetylenic and/or ethylenically unsaturated sites. Where carbon carbon double bonds exist, the configurational chemistry can be either cis (Z) or trans (E) and the depictions throughout the specification are not meant to be limiting. The depictions are, in general, presented based upon the configurational chemistry of related DHA or EPA compounds, and although not to be limited by theory, are believed to possess similar configuration chemistry.

Throughout the specification carbon carbon bonds in particular have been “distorted” for ease to show how the bonds may ultimately be positioned relative one to another. For example, it should be understood that acetylenic portions of the resolvins actually do include a geometry of approximately 180 degrees, however, for aid in understanding of the synthesis and relationship between the final product(s) and starting materials, such angles have been obfuscated to aid in comprehension.

It should be understood that hydrogenation of acetylenic portions of the resolvin analog may result in one or more products.

It is intended that all possible products are included within this specification. For example, hydrogenation of a diacetylenic resolvin analog can produce up to 8 products (four diene products, i.e., cis, cis; cis, trans; trans, cis; trans, trans) if hydrogenation of both acetylenic portions is completed (this can be monitored by known methods) and four monoacetylene-monoethylene products (cis or trans “monoene”—acetylene; acetylene-cis or trans “monoene”. All products can be separated and identified by HPLC, GC, MS, NMR, IR.

Known techniques in the art can be used to convert the carboxylic acid/ester functionality of the resolvin analog into carboxamides, thioesters, nitrile, carbamates, thiocarbamates, etc. and are incorporated herein. The appropriate moieties, such as amides, can be further substituted as is known in the art.

In general, the resolvin analogs of the invention are bioactive as alcohols. Enzymatic action or reactive oxygen species attack at the site of inflammation or degradative metabolism. Such interactions with the hydroxyl(s) of the resolvin molecule can eventually reduce physiological activity as depicted below:

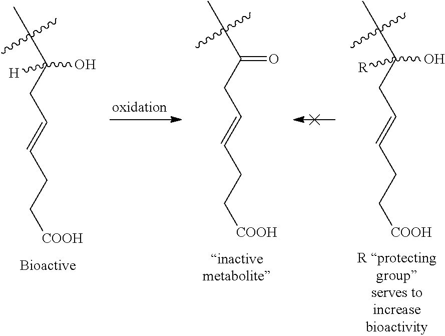

The use of “R” groups with secondary bioactive alcohols, in particular, serves to increase the bioavailability and bioactivity of the resolvin analog by inhibiting or diminishing the potential for oxidation of the alcohol to a ketone producing an inactive metabolite. The R “protecting groups” include, for example, linear and branched, substituted and unsubstituted alkyl groups, aryl groups, alkylaryl groups, phenoxy groups, and halogens.

Generally the use of “R protection chemistry” is not necessary with vicinal diols within the resolvin analog. Typically vicinal diols are not as easily oxidized and therefore, generally do not require such protection by substitution of the hydrogen atom adjacent to the oxygen atom of the hydroxyl group. Although it is generally considered that such protection is not necessary, it is possible to prepare such compounds where each of the vicinal diol hydroxyl groups, independently, could be “protected” by the substitution of the hydrogen atom adjacent to the oxygen atom of the hydroxyl group with an “R protecting group” as described above.

In another aspect, the invention provides other resolvins that are non-naturally occurring structural analogs of trihydroxy polyunsaturated eicosanoids as described in US Publication 2003/0236423, U.S. Ser. No. 10/405,924, filed Apr. 1, 2003 and published Dec. 25, 2003 by Nicos Petasis, the contents of which are incorporated herein in their entirety for all purposes. The synthetic polyunsaturated eicosanoids may exhibit improved chemical and biological properties. These include the compounds shown below, having the general formulas:

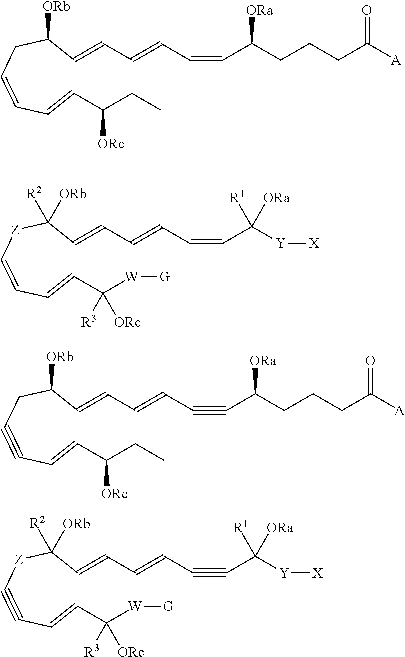

wherein,

A is hydroxy, alcoxy, aryloxy, amino, alkylamino, dialkylamino, or —OM, where M is a cation selected from a group consisting of ammonium, tetra-alkyl ammonium, Na, K, Mg, or Zn;

Ra, Rb and Rc, are independently selected from a group that consists of hydrogen, alkyl, aryl, heteroaryl, acyl, silyl, alcoxyacyl or aminoacyl;

R¹, R² and R³ are independently selected from a group that consists of hydrogen, alkyl, aryl or heteroaryl;

X is selected from a group that consists of:

—C(O)-A, —SO₂-A, —PO(OR)-A, where A is hydroxy, alcoxy, aryloxy, amino, alkylamino, dialkylamino, or —OM, where M is a cation selected from a group consisting of ammonium, tetra-alkyl ammonium, Na, K, Mg, or Zn; and R is a hydrogen or alkyl;

Y, Z and W are linkers selected from a group consisting of a ring or a chain of up to 20 atoms that may include one or more nitrogen, oxygen, sulfur or phosphorous atoms, provided that linker A can have one or more substituents selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that the linker may also contain one or more fused rings, including carbocyclic, heterocyclic, aryl or heteroaryl rings, provided that all linkers Y are connected to the adjacent C(R)OR group via a carbon atom;

G is selected from a group that consists of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.

In another aspect, the invention provides other resolvins that are non-naturally occurring structural analogs of trihydroxy polyunsaturated eicosanoids as described in US Publication 2004/0044050, U.S. Ser. No. 10/460,913, filed Jun. 13, 2003 and published Mar. 4, 2004 by Daniel Goodman et al., the contents of which are incorporated herein in their entirety for all purposes.

In one embodiment, the resolvin compounds are trihydroxy eicosapentaenoic acid analogues of a natural 5,12,18R-triHEPE. These include compounds of structural formula:

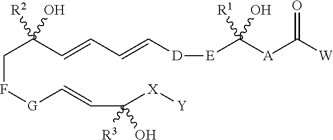

and pharmaceutically acceptable salts, hydrates and solvates thereof, wherein:

D-E and F-G are independently are cis or trans —C═C— or

—C≡C—

R¹, R² and R³ are independently selected from the group consisting of hydrogen, (C1-C4) straight-chained or branched alkyl, (C1-C4) alkoxy, —CH₂R⁴, —CHR⁴R⁴ and —CR⁴R⁴R⁴;

each R⁴ is independently selected from the group consisting of CN, —NO₂ and halogen;

W is selected from the group consisting of —R⁵, —OR⁵, —SR⁵ and —NR⁵R⁵;

each R⁵ is independently selected from the group consisting of hydrogen, (C1-C6) alkyl optionally substituted with one or more of the same or different R groups, (C5-C14) aryl optionally substituted with one or more of the same or different R groups, phenyl optionally substituted with one or more of the same or different R groups, (C6-C16) arylalkyl optionally substituted with one or more of the same or different R groups, 5-14 membered heteroaryl optionally substituted with one or more of the same or different R groups, 6-16 membered heteroarylalkyl optionally substituted with one or more of the same or different R groups and a detectable label molecule;

A is selected from the group consisting of (C1-C6) alkylene optionally substituted with 1, 2, 3, 4, 5 or 6 of the same or different halogen atoms, —(CH₂)ₙ—O—CH₂— and —(CH₂)ₘ—S—CH₂—, where m is an integer from 0 to 4;

X is selected from the group consisting of—(CH₂)ₙ— and —(CH₂), —O—, where n is an integer from 0 to 6;

Y is selected from the group consisting of hydrogen, (C1-C6) alkyl optionally substituted with one or more of the same or different R groups, (C5-C14) aryl optionally substituted with one or more of the same or different R groups, phenyl, optionally substituted with one or more of the same or different R groups, (C6-C16) arylalkyl optionally substituted with one or more of the same or different R groups, 5-14 membered heteroaryl optionally substituted with one or more of the same or different R groups, 6-16 membered heteroarylalkyl optionally substituted with one or more of the same or different R groups and a detectable label molecule;

each R is independently selected from the group consisting of an electronegative group, ═O, —OR^{a}, (C1-C3) haloalkyloxy, ═S, —SR^{a}, ═NR^{a}, ═NONR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)₂NR^{c}R^{c}, —OS(O)R^{a}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c}, —OC(O)R^{a}, —OC(O)OR^{a}, —OC(O)NR^{c}R^{c}, —OC(NH)NR^{c}R^{c}, —NHC(O)R^{a}, NHC(O)OR^{a}, —NHC(O)NR^{c}R^{c} and —NHC(NH)NR^{c}R^{c};

each R^{a} is independently selected from the group consisting of hydrogen and (C1-C4) alkyl; and

each R^{c} is independently an R^{a} or, alternatively, R^{c}R^{c} taken together with the nitrogen atom to which it is bonded forms a 5 or 6 membered ring.

The term “tissue” is intended to include intact cells, blood, blood preparations such as plasma and serum, bones, joints, muscles, smooth muscles, and organs.

The term “subject” is intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one of the compounds described herein, in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention. Such solutions are useful for the treatment of conjunctivitis.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Intravenous injection administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, epidural, intraorbital, intracardiac, intradermal, intraperitoneal, transtracheal, subcutaneous, subcuticular, intraarticular, subcapsular, subarachnoid, intraspinal and intrasternal injection and infusion.

In one particular aspect, epidural injection of the compounds of the invention into the epidural space can be used to treat or prevent neuropathic and/or inflammatory pain.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 0.1 to about 40 mg per kg per day. For example, between about 0.01 microgram and 20 micrograms, between about 20 micrograms and 100 micrograms and between about 10 micrograms and 200 micrograms of the compounds of the invention are administered per 20 grams of subject weight.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the compounds disclosed herein. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the compound disclosed hereinmay vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the EPA or DHA analog and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a compound disclosed herein is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Delivery of a compound disclosed herein to the lung by way of inhalation is an important method of treating a variety of respiratory conditions (airway inflammation) noted throughout the specification, including such common local conditions as bronchial asthma and chronic obstructive pulmonary disease. The compounds as disclosed herein can be administered to the lung in the form of an aerosol of particles of respirable size (less than about 10 μm in diameter). The aerosol formulation can be presented as a liquid or a dry powder. In order to assure proper particle size in a liquid aerosol, as a suspension, particles can be prepared in respirable size and then incorporated into the suspension formulation containing a propellant. Alternatively, formulations can be prepared in solution form in order to avoid the concern for proper particle size in the formulation. Solution formulations should be dispensed in a manner that produces particles or droplets of respirable size.

Once prepared an aerosol formulation is filled into an aerosol canister equipped with a metered dose valve. The formulation is dispensed via an actuator adapted to direct the dose from the valve to the subject.

Formulations of the invention can be prepared by combining (i) at least one of the compounds disclosed herein in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

Aerosol canisters equipped with conventional valves, preferably metered dose valves, can be used to deliver the formulations of the invention. Conventional neoprene and buna valve rubbers used in metered dose valves for delivering conventional CFC formulations can be used with formulations containing HFC-134a or HFC-227. Other suitable materials include nitrile rubber such as DB-218 (American Gasket and Rubber, Schiller Park, Ill.) or an EPDM rubber such as Vistalon™ (Exxon), Royalene™ (UniRoyal), bunaEP (Bayer). Also suitable are diaphragms fashioned by extrusion, injection molding or compression molding from a thermoplastic elastomeric material such as FLEXOMER™ GERS 1085 NT polyolefin (Union Carbide).

Formulations of the invention can be contained in conventional aerosol canisters, coated or uncoated, anodized or unanodized, e.g., those of aluminum, glass, stainless steel, polyethylene terephthalate.

The formulation(s) of the invention can be delivered to the respiratory tract and/or lung by oral inhalation in order to effect bronchodilation or in order to treat a condition susceptible of treatment by inhalation, e.g., asthma, chronic obstructive pulmonary disease, etc. as described throughout the specification.

The formulations of the invention can also be delivered by nasal inhalation as known in the art in order to treat or prevent the respiratory conditions mentioned throughout the specification.

While it is possible for a compound of the present invention to be administered alone, it is preferable to administer the compound as a pharmaceutical composition.

The invention features an article of manufacture that contains packaging material and a compound disclosed herein contained within the packaging material. This formulation contains an at least one compound disclosed herein and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference. Suitable compounds are described herein.

More specifically, the invention features an article of manufacture that contains packaging material and at least one compound described herein contained within the packaging material. The packaging material contains a label or package insert indicating that the formulation can be administered to the subject to asthma in an amount, at a frequency, and for a duration effective treat or prevent symptoms associated with such disease states or conditions discussed throughout this specification.

The following paragraphs enumerated consecutively from (1) through thirteen (13) provide for various aspects of the present invention. In one embodiment, in a first paragraph (1), the present invention provides a method to treat neuropathic pain, comprising the step of administering to a subject in need thereof an effective amount of a resolvin, such that neuropathic pain is treated.

2. The method of paragraph 1, wherein the neuropathic pain is associated with a disease condition selected from diabetic neuropathy or HIV infection.

3. A method to prevent the development of neuropathic pain, comprising the step of administering to a subject in need thereof an effective amount of a resolvin, such that neuropathic pain is prevented.

4. The method of paragraph 3, wherein the resolvin is administered during a surgical procedure, immediately after a spinal cord injury or after a stroke.

5. The method of paragraph 4, wherein the surgical procedure is a thoracotomy.

6. A method to treat post-operative pain, comprising the step of administering to a subject in need thereof an effective amount of a resolvin to treat post-operative pain after surgery.

7. A method to treat inflammatory pain, comprising the step of administering to a subject in need thereof an effective amount of a resolvin, such that the inflammatory pain is treated.

8. The method of paragraph 7, wherein the inflammatory pain is associated with arthritis pain, dental pain (e.g., TMJ), lower back pain, or inflammatory bowel disease. Low back pain contains mainly inflammatory and some neuropathic pain component.

9. A method to treat pain associated with cancer, comprising the step of administering to a subject in need thereof an effective amount of a resolvin, such that the pain associated with cancer is treated.

10. The method of paragraph 9, wherein the cancer is bone cancer.

11. The method of either of paragraphs 9 or 10, wherein the pain is characterized by both inflammatory and neuropathic pain components.

12. A method to treat pain associated with fibromyalgia syndrome, comprising the step of administering to a subject in need thereof an effective amount of a resolvin, such that the pain associated with fibromyalgia syndrome is treated.

13. The method of any of paragraphs 1 through 12, wherein analogues of RvE1 and RvD1, such as 19-(p-fluorophenoxy)-RvE1 (19-pf-RvE1) can be used to treat all the pain conditions mentioned above.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

Animals and pain models. Adult CD1 mice (male, 25-32 g) were used for most experiments. Knockout mice were used lacking TNFR1/II (Tnfrsf1a/1b^{-/-}) and TRPV1 (Trpv1^{-/-}) and C57BL/6 wild-type control mice, all from Jackson, for some experiments. All animal procedures performed in this study were approved by the Harvard Medical Area Standing Committee on Animals. 20 μl of diluted formalin (5%), carrageenan (CRG, 1%), or complete Freund's adjuvant (CFA) were injected into the plantar surface of a hindpaw to produce acute, persistent, or chronic inflammatory pain, respectively. Postoperative pain was also produced through hindpaw incision³⁶ and neuropathic pain by ligation of the spinal nerve³⁷.

Reagents and administration. RvE1 and RvD1 were suspended in 1% or 10% ethanol as vehicle for investigating acute (<1 h) and persistent (1-6 h) effects of resolvins on inflammatory pain. The reagents (10 μl) were delivered to the cerebral spinal fluid via intrathecal injection made by a spinal cord puncture between the L5 and L6 level with a 30G needle³⁸.

Immunohistochemistry. Mice were deeply ananesthetized with isoflurane and perfused mice through the ascending aorta with 4% paraformaldehyde. The spinal cord (L4-L5 segment) and DRG (L4, L5) tissues were removed and postfixed spinal cord overnight and DRG 2 h. Spinal cord sections were cute (30 μm, free-floating) and DRG sections (14 p.m) in a cryostat and performed immunofluorescence. The sections were blocked with 2% goat or donkey serum for 1 h at room temperature, and incubated the sections with primary antibodies overnight at 4° C., then with Cy3- or FITC-conjugated secondary antibodies (1:400, Jackson immunolab) for 1 h at room temperature. For double immunofluorescence, tissue sections were incubated with a mixture of polyclonal and monoclonal primary antibodies followed by a mixture of FITC- and CY3-congugated secondary antibodies. ChemR23 was amplified by staining with TSA (Tyramide Signal Amplification) system.

Primary DRG culture. DRGs were aseptically removed from 4-week old mice and digested them first with collagenase (1.25 mg/ml)/dispase-II (2.4 units/ml) at 37° C. for 90 min then with 0.25% trypsin for 8 min at 37° C. DRG cells were mechanically dissociated with a flame polished Pasteur pipette in the presence of 0.05% DNAse I and plated these cells onto poly-D-lysine and laminin-coated slide chambers. DRG cells were grown in a neurobasal defined medium (with 2% B27 supplement, Invitrogen) in the presence of NGF (50 ng/ml) for 24 h and replaced the medium with NGF-free medium before stimulation. For immunocytochemistry, DRG cells were fixed with 4% paraformaldehyde for 30 min and incubated the cells with pERK primary antibody (rabbit, Cell signaling, 1:500) overnight.

Spinal cord slice preparation. As previously reported³⁹, a portion of the lumbar spinal cord (L4-L5) was removed from young mice (3-4 week old) under urethane anesthesia (1.5-2.0 g/kg, i.p.) and transverse spinal cord slices were cut (600 μm) on a vibrating microslicer. The slices were perfused with Kreb's solution (8 ml/min) for >2 h prior to experiments. Some slices were stimulated with TNF-α (10 ng/ml, 5 min), the slices were fixed with 4% paraformaldehyde for 1 h, and processed pERK immunohistochemistry using thin sections (15 μm)¹³.

Patch clamp recordings in spinal slices. Whole cell patch-clamp recordings were performed in lamina II neurons in voltage clamp mode³⁹. After establishing the whole-cell configuration, neurons were held at the holding potentials of −70 mV for recording spontaneous excitatory postsynaptic current (sEPSC). NMDA-induced currents were recorded by bath application of NMDA (50 μM, Sigma) at the holding potential of −50 mV. Membrane currents were amplified with an Axopatch 200A amplifier (Axon Instruments) in voltage-clamp mode. pCLAMP 6 was used and Mini Analysis (Synaptosoft Inc.) software to store and analyze the data. Those cells showing >5% changes from the baseline levels were reagrded as responding ones²⁴.

Behavioral analysis. Mice were habituated in the testing environment for two days and performed behavioral testing in a blinded manner. Formalin-evoked spontaneous inflammatory pain was assessed by measuring duration (seconds) animals spent on licking/flinching the affected paws every 5 min for 45 min. Capsaicin-induced spontaneous pain was aslo assessed for 10 min. For testing mechanical sensitivity, mice were put in boxes on an elevated metal mesh floor and stimulated hindpaw with a series of von Frey hairs with logarithmically incrementing stiffness (0.02-2.56 grams, Stoelting), presented perpendicular to the plantar surface. It was determined the 50% paw withdrawal threshold using Dixon's up-down method⁴⁰. For testing heat sensitivity, mice were placed in plastic boxes and tested heat sensitivity using Hargreaves radiant heat apparatus (IITC Life Science Inc.). The basal paw withdrawal latency was adjusted to 9-12 seconds and set a cut-off of 20 seconds to prevent tissue damage. The percent maximal possible antinociceptive effect (% M.P.E) was calculated using the equation % M.P.E.=[(PL−BL2)/(BL1−BL2)]×100. BL1, baseline latency before inflammation; BL2, baseline latency after inflammation but before drug injection; PL, latency post drug injection.

Statistics. Data was expressed as mean±SEM and compared the differences between groups using student t-test or ANOVA followed by Newman-Keuls test. The criterion for statistical significance was P<0.05.

### REFERENCE LIST

1. Schnitzer, T. J. Update on guidelines for the treatment of chronic musculoskeletal pain. Clin. Rheumatol. 25 Suppl 1, S22-S29 (2006).
2. Serhan, C. N., Chiang, N., & Van Dyke, T. E. Resolving inflammation: dual anti-inflammatory and pro-resolution lipid mediators. Nat. Rev. Immunol. 8, 349-361 (2008).
3. Connor, K. M. et al. Increased dietary intake of omega-3-polyunsaturated fatty acids reduces pathological retinal angiogenesis. Nat. Med. 13, 868-873 (2007).
4. Gilroy, D. W. et al. Inducible cyclooxygenase may have anti-inflammatory properties. Nat. Med. 5, 698-701 (1999).
5. Schwab, J. M., Chiang, N., Arita, M., & Serhan, C. N. Resolvin E1 and protectin D1 activate inflammation-resolution programmes. Nature 447, 869-874 (2007).
6. Chiang, N. et al. Anesthetics impact the resolution of inflammation. PLoS. ONE. 3, e1879 (2008).
7. Haworth, O., Cernadas, M., Yang, R., Serhan, C. N., & Levy, B. D. Resolvin E1 regulates interleukin 23, interferon-gamma and lipoxin A4 to promote the resolution of allergic airway inflammation. Nat. Immunol. 9, 873-879 (2008).
8. Julius, D. & Basbaum, A. I. Molecular mechanisms of nociception. Nature 413, 203-210 (2001).
9. Hucho, T. & Levine, J. D. Signaling pathways in sensitization: toward a nociceptor cell biology. Neuron 55, 365-376 (2007).
10. Woolf, C. J. & Salter, M. W. Neuronal plasticity: increasing the gain in pain. Science 288, 1765-1769 (2000).
11. Ji, R. R., Kohno, T., Moore, K. A., & Woolf, C. J. Central sensitization and LTP: do pain and memory share similar mechanisms? Trends Neurosci. 26, 696-705 (2003).
12. Yamamoto, T. & Yaksh, T. L. Comparison of the antinociceptive effects of pre- and posttreatment with intrathecal morphine and MK801, an NMDA antagonist, on the formalin test in the rat. Anesthesiology 77, 757-763 (1992).
13. Ji, R. R., Baba, H., Brenner, G. J., & Woolf, C. J. Nociceptive-specific activation of ERK in spinal neurons contributes to pain hypersensitivity. Nat. Neurosci. 2, 1114-1119 (1999).
14. Yaksh, T. L. Spinal systems and pain processing: development of novel analgesic drugs with mechanistically defined models. Trends Pharmacol. Sci. 20, 329-337 (1999).
15. Kawasaki, Y. et al. Distinct roles of matrix metalloproteases in the early- and late-phase development of neuropathic pain. Nat. Med. 14, 331-336 (2008).
16. Arita, M. et al. Stereochemical assignment, antiinflammatory properties, and receptor for the omega-3 lipid mediator resolvin E1. J. Exp. Med. 201, 713-722 (2005).
17. Cash, J. L. et al. Synthetic chemerin-derived peptides suppress inflammation through ChemR23. J. Exp. Med. 205, 767-775 (2008).
18. Wittamer, V. et al. Specific recruitment of antigen-presenting cells by chemerin, a novel processed ligand from human inflammatory fluids. J. Exp. Med. 198, 977-985 (2003).
19. Liu, X. J. et al. Treatment of inflammatory and neuropathic pain by uncoupling Src from the NMDA receptor complex. Nat. Med. 14, 1325-1332 (2008).
20. Goldberg, R. J. & Katz, J. A meta-analysis of the analgesic effects of omega-3 polyunsaturated fatty acid supplementation for inflammatory joint pain. Pain 129, 210-223 (2007).
21. Arita, M. et al. Metabolic inactivation of resolvin E1 and stabilization of its anti-inflammatory actions. J. Biol. Chem. 281, 22847-22854 (2006).
22. Schafers, M., Svensson, C. I., Sommer, C., & Sorkin, L. S. Tumor necrosis factor-alpha induces mechanical allodynia after spinal nerve ligation by activation of p38 MAPK in primary sensory neurons. J. Neurosci. 23, 2517-2521 (2003).
23. Junger, H. & Sorkin, L. S. Nociceptive and inflammatory effects of subcutaneous TNFalpha. Pain 85, 145-151 (2000).
24. Kawasaki, Y., Zhang, L., Cheng, J. K., & Ji, R. R. Cytokine mechanisms of central sensitization: distinct and overlapping role of interleukin-1beta, interleukin-6, and tumor necrosis factor-alpha in regulating synaptic and neuronal activity in the superficial spinal cord. J. Neurosci 28, 5189-5194 (2008).
25. Caterina, M. J. et al. Impaired nociception and pain sensation in mice lacking the capsaicin receptor. Science 288, 306-313 (2000).
26. Nicol, G. D., Lopshire, J. C., & Pafford, C. M. Tumor necrosis factor enhances the capsaicin sensitivity of rat sensory neurons. J. Neurosci 17, 975-982 (1997).
27. Jin, X. & Gereau, R. W. Acute p38-mediated modulation of tetrodotoxin-resistant sodium channels in mouse sensory neurons by tumor necrosis factor-alpha. J. Neurosci 26, 246-255 (2006).
28. Zhuang, Z. Y., Xu, H., Clapham, D. E., & Ji, R. R. Phosphatidylinositol 3-kinase activates ERK in primary sensory neurons and mediates inflammatory heat hyperalgesia through TRPV1 sensitization. J. Neurosci 24, 8300-8309 (2004).
29. Vara, H., Onofri, F., Benfenati, F., Sassoe-Pognetto, M., & Giustetto, M. ERK activation in axonal varicosities modulates presynaptic plasticity in the CA3 region of the hippocampus through synapsin I. Proc. Natl. Acad. Sci. U. S. A 106, 9872-9877 (2009).
30. Karim, F., Wang, C. C., & Gereau, R. W. Metabotropic glutamate receptor subtypes 1 and 5 are activators of extracellular signal-regulated kinase signaling required for inflammatory pain in mice. J. Neurosci. 21, 3771-3779 (2001).
31. Samad, T. A. et al. Interleukin-1beta-mediated induction of Cox-2 in the CNS contributes to inflammatory pain hypersensitivity. Nature 410, 471-475 (2001).
32. Woolf, C. J. Mu and delta opioid receptors diverge. Cell 137, 987-988 (2009).
33. Porreca, F. & Ossipov, M. H. Nausea and Vomiting Side Effects with Opioid Analgesics during Treatment of Chronic Pain: Mechanisms, Implications, and Management Options. Pain Med. (2009).
34. Funk, C. D. & FitzGerald, G. A. COX-2 inhibitors and cardiovascular risk. J. Cardiovasc. Pharmacol. 50, 470-479 (2007).
35. Gavva, N. R. Body-temperature maintenance as the predominant function of the vanilloid receptor TRPV1. Trends Pharmacol. Sci. 29, 550-557 (2008).
36. Brennan, T. J., Vandermeulen, E. P., & Gebhart, G. F. Characterization of a rat model of incisional pain. Pain 64, 493-501 (1996).
37. Kim, S. H. & Chung, J. M. An experimental model for peripheral neuropathy produced by segmental spinal nerve ligation in the rat. Pain 50, 355-363 (1992).
38. Hylden, J. L. & Wilcox, G. L. Intrathecal morphine in mice: a new technique. Eur. J. Pharmacol. 67, 313-316 (1980).
39. Gao, Y. J. et al. JNK-induced MCP-1 production in spinal cord astrocytes contributes to central sensitization and neuropathic pain. J. Neurosci. 29, 4096-4108 (2009).
40. Dixon, W. J. Efficient analysis of experimental observations. Annu. Rev. Pharmacol. Toxicol. 20, 441-462 (1980).

Preparation and Delivery of Resolvins

Resolvins (RvD1 and RvE1) were initially isolated in exudates formed in the resolution phase of acute inflammation and were then found in human plasma. After the full structural elucidation, resolvins were produced by full organic chemical synthesis^{1,2}. Synthetic RvE1 and RvD1 were obtained from Cayman Chemical (MI) and were qualified according to published physical and biological properties^{2,3}. The RvE1 analog was prepared and qualified as previously shown⁴. The stock solution contained 10 or 100 ng/μl resolvin suspended in 100% ethanol and was kept in a −80° C. freezer. Caution was taken to avoid exposure of resolvins to air during the preparation for injection. 1% ethanol was used as vehicle for testing acute effects on pain behaviors (<1 h, FIG. 1a-d, 2b, 3f; Supplementary FIG. 1a, 1b, 5a, 5b). 10% ethanol was also used as vehicle for testing sustained effects on pain behaviors (>1 h, FIG. 2c-e, 3c, 4a, Supplementary FIG. 1d, 7b-d).

Intrathecal injection. To avoid systemic effects of drugs and target spinal mechanisms, drugs were delivered into cerebral spinal fluid (CSF) space around lumbosacral spinal cord via intrathecal (i.t.) administration. Spinal cord puncture was made with a 30G needle between the L5 and L6 level to deliver the reagents (10 μl) to the cerebral spinal fluid (Hylden and Wilcox, 1980)⁵. A successful spinal puncture was evidenced by a brisk tail-flick after the needle entry into subarachnoid space (change in resistance). >95% accuracy was achieved by a dye injection. In addition to spinal cord cells, intrathecal drug administration also affects DRG cells^{6,7}.

Effects of ethanol on pain sensitivity: It was found that 1% ethanol (10 μl, i.t.) did not alter CFA-induced heat hyperalgesia when tested at 15, 30, and 45 min after the injection (FIG. 2b). However, 10% ethanol (10 μl) produced mild and transient inhibition on pain at 30 min (data not shown) but not at 1 h (FIG. 2d). Therefore, to avoid the acute influence of 10% ethanol, pain behavior was tested one hour after the drug injection with this vehicle (FIG. 2c). The anti-hyperalgesic efficacy of RvE1 prepared in 1% and 10% ethanol was compared on post-CFA day 3. At 1 h after the RvE1 injection, RvE1 (10 ng, i.t.) produced similar anti-hyperalgesic effects with two different vehicles. There were 54.7% and 55.7% reduction of heat hyperalgesia in 1% and 10% ethanol, respectively (P=0.92, n=6). Thus, 1% ethanol has no effect on pain behavior in all the time points tested, and 10% ethanol only affects acute pain behavior and but has no effect on pain behavior when tested 0.5 (FIG. 2d) or 1 h (FIG. 2c) after the injection.

Resolvins were also used for intraplantar injection. RvD1 or RvE1 was injected into the plantar surface of a hindapaw [20 ng, 20 μl , in 1% ethanol (Supplementary FIG. 5a, 5b) or in 10% ethanol (FIG. 2e, Supplementary FIG. 1d)]. DRG cultures were incubated with RvE1 (10 ng/ml, 0.1% ethanol in culture medium) and perfused spinal cord slices with RvE1 (1 ng/ml, in 0.01% ethanol and Kreb's solution).

Small Interfering RNAs (siRNAs) and Delivery

The ChemR23 siRNA and its non-targeting control siRNA were synthesized by Dharmacon according to the published sequences8:

(1) ChemR23 siRNA: 5′-AACACUGUGUGGUUUGUCAACtt-3′;

(2) Control siRNA (non-targeting): 5′-GACUUCGCGGGACACAUGAtt-3′;

siRNAs were mixed with the transfection reagent polyethyleneimine (PEI, Fermentas), 10 min before injection, to increase cell membrane penetration and reduce the degradation (Tan et al., 2005; 2009)^{9,10}. PEI was dissolved in 5% glucose, and 1 pg of siRNA was mixed with 0.18 μl of PEI, as shown in our previous study (Kawasaki et al., 2008)⁷. In the formalin model, siRNA was administrated via intrathecal route (3 μg), delivered at 72 h, 48 h, and 24 h before formalin injection (supplementary FIG. 1a, b). In the carrageenan model, siRNA was administrated via intraplantar route (4 μg), delivered at 48 h, 24 h, and 3 h before carrageenan injection (supplementary FIG. 1c, d). The knockdown effects of ChemR23 siRNA were examined by Western blotting using a ChemR23 antibody (Santa Cruz).

Other Drugs and Delivery

The following drugs were used for intrathecal injection, intraplantar injection, DRG culture incubation, and spinal cord slice perfusion.

Intrathecal injection: DHA and EPA (Cayman Chemical, 10 μg, prepared in 1% ethanol), COX-2 inhibitor NS-398 (Sigma, 0.1, 1, and 10 μg, prepared in 1% ethanol in FIG. 1a-c or in 10% ethanol in FIG. 2c), morphine (Sigma, 10 to 500 ng, prepared in 1% ethanol in FIG. 1a-c), naloxone (Sigma, 2 μg, prepared in 1% ethanol), pertussis toxin (Tocris Bioscience, 0.2 μg, prepared in saline), chemerin (R & D Systems, 0.45 to 100 ng, prepared in 1% ethanol), TNF-α (R & D Systems, 20 ng, prepared in saline), and capsaicin (Sigma, 0.5 μg in 10 μl, prepared in 1% DMSO) were delivered. To examine whether pretreatment of RvE1 attenuated TNF-α-induced persistent hyperalgesia at 1, 3, and 24 h, RvE1 and TNF-α were mixed and injected at the same time (FIG. 3c). To examine whether pretreatment of RvE1 attenuates capsaicin-induced acute spontaneous pain, RvE1 and capsaicin were mixed and injected at the same time (FIG. 3f). Unlike other drugs, pertussis toxin was given 24 h prior to formalin test for single injection (1×0.2 μg) or 24 and 12 h prior to formalin test for double injections (2×0.2 μg).

Intraplantar injection: Capsaicin (Sigma, 5 μg in 5 μl , prepared in 10% DMSO) was injected into the plantar surface of a hindpaw. To examine whether pretreatment of resolvins attenuates pain in several conditions, resolvins were administrated 10 min before the injection of formalin, capsaicin, or carrageenan.

Incubation of DRG cultures: Culture DRG neurons were incubated with TNF-α (10 ng/ml, in culture medium), capsaicin (500 nM, in 0.01% DMSO and culture medium), and pertussix toxin (0.2 μg/ml, in culture medium). For pertussix toxin, DRG cultures were treated with the toxin for 24 h before stimulation.

Perfusion of spinal cord slices: Spinal cord slices were incubated with chemerin (100 ng/ml, in Kreb's solution), TNF-α (10 ng/ml, in Kreb's solution), capsaicin (100 nM, in 0.01% DMSO and Kreb's solution), TRPV1 antagonist capsazepine (Sigma, 10 μM, in 0.01% DMSO and Kreb's solution), pertussix toxin (0.5 μg/ml, in Kreb's solution), and MEK inhibitor PD98059 and U0126 (CalBiochem, 1 μM, in 0.01% DMSO and Kreb's solution). For pertussix toxin treatment, spinal cord slices were incubated with the toxin for 3 h before stimulation.

Primary Antibodies Used for Immunohistochemistry and Immunocytochemistry

The following primary antibodies were used: TRPV1 antibody (rabbit, 1:500, Chemicon), TRPV1 antibody (guinea pig, 1:1000, Chemicon), Substance P antibody (guinea pig, 1:1000, Chemicon), NeuN antibody (mouse, 1:5000, Chemicon), and phosphorylated ERK antibody (pERK1/2, rabbit, 1:300, Cell Signaling). These antibodies have been extensively tested in the literature, and the present staining patterns are identical to that of previous reports. The specificity of immunostaining was tested by absence of staining after omitting the primary antibody.

Two different ChemR23 antibodies were used, a goat antibody recognizing an N-terminal extracellular domain of ChemR23 (1:200, Santa Cruz) and a rabbit antibody recognizing internal region (aa 151-310) of ChemR23 (1:500, Santa Cruz). These two different antibodies showed similar staining patterns in the DRG and spinal cord. They also showed similar patterns in mouse and rat tissues. To further test the specificity of ChemR23 antibody, a blocking peptide (with a sequence mapping within an N-terminal extracellular domain of ChemR23, Santa Cruz) was obtained that was used to produce ChemR23 antibody. The ChemR23 antibody (Goat, 1:200) failed to produce positive staining after incubation with the blocking peptide (5 μM) overnight at 4° C. (Supplementary FIG. 3c).

Tyramide Signal Amplification of Immunostaining

To enhance the signal of immunostaining, TSA (Tyramide Signal Amplification) kit (Perkin Elmer, Mass.) was used for some tissue sections. In brief, after the primary antibody incubation, the sections were incubated with a biotinylated-secondary antibody (1:400, 1 h at room temperature), followed by avidin-streptin incubation (1:100, 1 h at room temperature), and finally by tyramide incubation (1:50, 5 min at room temperature).

Quantification of Immunohistochemistry and Immunocytochemistry

pERK immunocytochemistry in DRG neurons: DRG cells grown in slide chambers were fixed with 4% paraformaldehyde for 30 min and incubated with pERK primary antibody (Rabbit, Cell Signaling, 1:500) at 4° C. overnight. The stained cells were examined with a Nikon fluorescence microscope, and images in 3 randomly-selected optic fields from each chamber were captured with a CCD Spot camera. The number of pERK-positive neurons and the total number of neurons in each field were counted, and the percentage of pERK-positive neurons in each field was calculated as previously demonstrated¹¹. The data from three fields were averaged for each culture and 4 separate cultures were included for data analysis.

PERK immunohistochemistry in the spinal cord: Transverse spinal cord slices (600 μm) were cut on a vibrating microslicer. After treatment, spinal cord slices were fixed with 4% paraformaldehyde for 60 min and incubated in 20% sucrose over night at 4° C. The spinal slices were then cut into thin sections (15 μm) in cryostat. Five spinal cord sections were randomly selected for pERK immunostaining using a rabbit antibody (Cell Signaling, 1:300). The stained sections were examined with a Nikon fluorescence microscope, and images were captured with a CCD Spot camera. The number of pERK-immunoreactive cells in the superficial dorsal horn (laminae I-III) was quantified in 5 spinal cord sections from each slice and averaged for that slice, and 4-5 slices from separate animals were included for data analysis¹². Previous studies have shown that pERK is predominantly induced in the superficial spinal cord following C-fiber activation¹³.

Assessment of Inflammation

Measurement of paw volume. Paw swelling (edema) after carrageenan injection was determined by water displacement plethysmometer (Ugo Basile, Italy). The Plethysmometer is a microcontrolled volume meter, specially designed for accurate measurement of the rat/mouse paw swelling. It consists of a water filled Perspex cell into which the paw is dipped. A transducer of original design records small differences in water level, caused by volume displacement. The digital read-out shows the exact volume of the paw.

Myeloperoxidase assay. Paw inflammation is associated with infiltration of neutrophils (polymorphonuclear cells), and myeloperoxidase (MPO) activity is often used to quantify neutrophil activation to evaluate the magnitude of paw inflammation. To determine MPO assay, fresh skin tissues were suspended in phosphate buffer and homogenized. The suspension was centrifuged and the supernatant was assayed for MPO activity using a MPO Activity Assay Kit (Northwest Life Science Specialties, LLC). Each reaction was performed according to manufacturer's protocol. Briefly, hypochlorous acid (HOCl) is formed from MPO catalyzed reaction between chloride and hydrogen peroxide. HOCl is rapidly trapped by β-amino acid taurine to form a stable oxidant taurine chloramine. Taurine chloramine is then allowed to react with 5-thio-2-nitrobenzoic acid (TNB). TNB has a chromophore that has maximal absorbance at 412 nm while its reaction product with taurin chloramine is colorless. The color change is detected by spectrophotometry. By following the decrease of absorbance at 412 nm, MPO activity is measured. One unit is the amount of MPO that can produce 1.0 nmole of taurine chloramine at pH 6.5 and 25° C. during 30 minutes in the presence of 100 mM chloride and 100 μM of hydrogen peroxide.

Western Blotting

Animals were transcardially perfused with PBS, and the skin, DRG, and spinal cord tissues were rapidly removed and homogenized in a lysis buffer containing a cocktail of protease inhibitors and phosphatase inhibitors. The protein concentrations were determined by BCA Protein Assay (Pierce), and 30 μg of proteins were loaded for each lane and separated on SDS-PAGE gel (4-15%, Bio-Rad). After the transfer, the blots were incubated overnight at 4° C. with polyclonal antibody against ChemR23 (goat, 1:200, Santa Cruz). For loading control, the blots were probed with tubulin antibody (mouse, 1:5000, Sigma).

Cytokine Array

The mouse cytokine array kits were purchased from R&D. Protein samples were prepared in the same way as for Western blotting analysis. Each reaction was performed according to manufacturer's protocol using 400 μg proteins. In brief, protein samples were incubated with a blot (array) that is pre-coated with 40 cytokines/chemokines for 24 h at 4° C. The remaining procedures were similar to that of Western blotting. Also see Gao et al., 2009¹².

In Situ Hybridization

Animals were transcardially perfused with PBS and 4% paraformaldehyde. DRG and spinal cord tissues were collected and post-fixed overnight. These tissues were sectioned at a thickness of 12 μm (DRG sections) and 15 μm (spinal cord sections) and mounted on Superfrost plus slides. The ChemR23 riboprobe (0.48 kb) was generated by PCR. The reverse primer contains T7 RNA polymerase binding sequence (CGATGTTAATACGACTCACTATAGGG) for the generation of the antisense riboprobe. DNA sequences were transcribed in vitro with T7 RNA polymerase (Promega) in the presence of digoxigenin-labeling mix. In situ hybridization was performed as previously described^{12,14}. Briefly, sections were hybridized with ChemR23 riboprobe (1 μg/ml) overnight at 65° C. After washing, sections were blocked with 20% serum for 1 h at room temperature followed by incubation with alkaline phosphatase-conjugated anti-digoxigen antibody (1:2000; Roche Diagnostics) overnight at 4° C. Sections were then incubated with a mixture of nitro-blue tetrazolium (NBT) and 5-bromo-4-chloro-3-indolyl-phosphate (BCIP) in alkaline phosphatase buffer for 24-48 h for color development. In situ images were captured with a Nikon microscope under bright-field.

### REFERENCE LIST

1. Serhan, C. N. et al. Resolvins: a family of bioactive products of omega-3 fatty acid transformation circuits initiated by aspirin treatment that counter proinflammation signals. J. Exp. Med. 196, 1025-1037 (2002).
2. Serhan, C. N., Chiang, N., & Van Dyke, T. E. Resolving inflammation: dual anti-inflammatory and pro-resolution lipid mediators. Nat. Rev. Immunol. 8, 349-361 (2008).
3. Arita, M. et al. Resolvin E1, an endogenous lipid mediator derived from omega-3 eicosapentaenoic acid, protects against 2,4,6-trinitrobenzene sulfonic acid-induced colitis. Proc. Natl. Acad. Sci. U.S. A 102, 7671-7676 (2005).
4. Arita, M. et al. Metabolic inactivation of resolvin E1 and stabilization of its anti-inflammatory actions. J. Biol. Chem. 281, 22847-22854 (2006).
5. Hylden, J. L. & Wilcox, G. L. Intrathecal morphine in mice: a new technique. Eur. J. Pharmacol. 67, 313-316 (1980).
6. Ji, R. R., Samad, T. A., Jin, S. X., Schmoll, R., & Woolf, C. J. p38 MAPK activation by NGF in primary sensory neurons after inflammation increases TRPV1 levels and maintains heat hyperalgesia. Neuron 36, 57-68 (2002).
7. Kawasaki, Y. et al. Distinct roles of matrix metalloproteases in the early- and late-phase development of neuropathic pain. Nat. Med. 14, 331-336 (2008).
8. Arita, M. et al. Stereochemical assignment, antiinflammatory properties, and receptor for the omega-3 lipid mediator resolvin E1. J. Exp. Med. 201, 713-722 (2005).
9. Tan, P. H., Yang, L. C., Shih, H. C., Lan, K. C., & Cheng, J. T. Gene knockdown with intrathecal siRNA of NMDA receptor NR2B subunit reduces formalin-induced nociception in the rat. Gene Ther. 12, 59-66 (2005).
10. Tan, P. H., Yang, L. C., & Ji, R. R. Therapeutic potential of RNA interference in pain medicine. Open. Pain 12, 57-63 (2009).
11. Schweizerhof, M. et al. Hematopoietic colony-stimulating factors mediate tumor-nerve interactions and bone cancer pain. Nat. Med. 15, 802-807 (2009).
12. Gao, Y. J. et al. JNK-induced MCP-1 production in spinal cord astrocytes contributes to central sensitization and neuropathic pain. J. Neurosci. 29, 4096-4108 (2009).
13. Kawasaki,Y. et al. Ionotropic and metabotropic receptors, protein kinase A, protein kinase C, and Src contribute to C-fiber-induced ERK activation and cAMP response element-binding protein phosphorylation in dorsal horn neurons, leading to central sensitization. J. Neurosci 24, 8310-8321 (2004).
14. Chen, C. L. et al. Runx1 determines nociceptive sensory neuron phenotype and is required for thermal and neuropathic pain. Neuron 49, 365-377 (2006).

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

1.-13. (canceled)

14. A method to treat neuropathic pain, comprising the step of administering to a subject in need thereof an effective amount of a resolvin, such that neuropathic pain is treated.

15. The method of claim 14, wherein the neuropathic pain is associated with a disease condition selected from diabetic neuropathy or HIV infection.

16. A method to prevent the development of neuropathic pain, comprising the step of administering to a subject in need thereof an effective amount of a resolvin, such that neuropathic pain is prevented.

17. The method of claim 16, wherein the resolvin is administered during a surgical procedure, immediately after a spinal cord injury or after a stroke.

18. The method of claim 17, wherein the surgical procedure is a thoracotomy.

19. A method to treat post-operative pain, comprising the step of administering to a subject in need thereof an effective amount of a resolvin to treat post-operative pain after surgery.

20. A method to treat inflammatory pain, comprising the step of administering to a subject in need thereof an effective amount of a resolvin, such that the inflammatory pain is treated.

21. The method of claim 20, wherein the inflammatory pain is associated with arthritis pain, dental pain (e.g., TMJ), lower back pain, or inflammatory bowel disease.

22. A method to treat pain associated with cancer, comprising the step of administering to a subject in need thereof an effective amount of a resolvin, such that the pain associated with cancer is treated.

23. The method of claim 22, wherein the cancer is bone cancer.

24. The method of either of claim 22, wherein the pain is characterized by both inflammatory and neuropathic pain components.

25. A method to treat pain associated with fibromyalgia syndrome, comprising the step of administering to a subject in need thereof an effective amount of a resolvin, such that the pain associated with fibromyalgia syndrome is treated.

26. The method of claim 14, wherein analogues of RvE1 and RvD1 can be used to treat neuropathic pain.

27. The method of claim 16, wherein analogues of RvE1 and RvD1 can be used to prevent the development of neuropathic pain.

28. The method of claim 19, wherein analogues of RvE1 and RvD1 can be used to treat post-operative pain.

29. The method of claim 20, wherein analogues of RvE1 and RvD1 can be used to treat inflammatory pain.

30. The method of claim 22, wherein analogues of RvE1 and RvD1 can be used to treat pain associated with cancer.

31. The method of claim 25, wherein analogues of RvE1 and RvD1 can be used to treat pain associated with bibromyalgia.
